(19) United States
(12) Patent Application Publication
(10) Pub. No.: US 2023/0285364 A1
(43) Pub. Date: Sep. 14, 2023

(54) USE OF A DHODH INHIBITOR COMPOUND IN COMBINATION CANCER THERAPY

(71) Applicant: Servier Pharmaceuticals LLC, Boston, MA (US)

(72) Inventors: John Coco, North Andover, MA (US); Joshua Murtie, Winchester, MA (US); Danielle Ulanet, Cambridge, MA (US)

(21) Appl. No.: 18/012,136

(22) PCT Filed: Jun. 23, 2021
(86) PCT No.: PCT/US2021/038741

§ 371 Date: Dec. 21, 2022

Related U.S. Application Data
(60) Provisional application No. 63/043,350, filed on Jun. 24, 2020.

(51) Int. Cl. A61K 31/4192; A61K 31/635; A61K 31/7068; A61K 31/706; A61P 35/02; A61P 35/00

(52) U.S. Cl. CPC A61K 31/4192; A61K 31/635; A61K 31/706; A61K 31/7068; A61P 35/00; A61P 35/02

(57) ABSTRACT

Provided herein are method of treating cancers using a first anti-cancer agent and at least one second anti-cancer agent, wherein the first anti-cancer agent is represented by Formula (I): or a pharmaceutically acceptable salt thereof. Compositions comprising the disclosed first anti-cancer agent and at least one second anti-cancer agent are also provided.

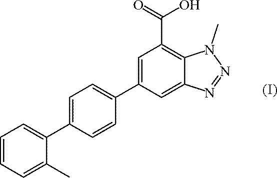 [SMILES: Cc1ccccc1-c1ccc(-c2cc(C(=O)O)c3c(c2)nnn3C)cc1]

### RELATED APPLICATION

This application claims priority to U.S. Provisional Application No. 63/043,350, filed on Jun. 24, 2020, the entire contents of which are expressly incorporated herein by reference in its entirety.

### BACKGROUND

Leukemia, lymphoma, myeloma and myelodysplastic syndromes (MDS) are types of cancer that can affect the bone marrow, the blood cells, the lymph nodes and other parts of the lymphatic system. In the United States, it is estimated that one person in every three minutes is diagnosed with a hematological cancer and one person in every nine minutes dies from it.

Of note, acute monocytic leukemia is a distinct subtype of acute myeloid leukemia (AML) with characteristic clinical features of a patient having >20% blasts in the bone marrow, of which >80% are of the monocytic lineage. Acute monocytic leukemia has been reported to have a poor prognosis compared to other subtypes of AML, and it has been shown that the disease may develop after chemotherapy exposure, particularly following epipodophyllotoxins and anthracyclines.

Acute lymphoblastic leukemia (ALL), another leukemia subtype, is an aggressive hematologic malignancy wherein an abnormal proliferation of lymphoblasts suppresses normal hematopoiesis resulting in progressive marrow failure and death. This particular subtype has a bimodal age distribution with an initial peak in childhood and second that increases in older adults. While outcomes for ALL in children have improved with modern chemotherapy regimens, outcomes in adults remain dismal, which are attributable to a combination of increased adverse tumor biology and decreased tolerance of therapy. As a result despite a high initial remission rate most adults are destined to relapse. It has been declared that most adults with recurrent ALL “cannot be rescued with current therapies”. This is likely the result of the fact that virtually all active therapies are used during first line treatment. There is clearly a need for additional active therapies in ALL.

A pioneering genetic evaluation of diffuse large B-cell lymphoma (DLBCL) reveals that the dual rearrangement of MYC and BCL2/BCL6 occurs in approximately 5% to 7% of patients with DLBCL. This form of DLBCL has recently been re-classified as “high-grade B-cell lymphoma, with MYC and BCL2 and/or BCL6 rearrangements”, and is now commonly referred to as “double hit DLBCL” or “double hit lymphoma”. Sometimes, all 3 genes - MYC, BCL2 and BCL6 - are simultaneously rearranged in a phenotype termed “triple hit DLBCL” or “triple hit lymphoma”. Both double hit and triple hit lymphomas are highly proliferative and drug-resistant. Both phenotypes are also associated with an extremely poor prognosis with standard treatment, such as rituximab plus cyclophosphamide, doxorubicin, vincristine, and prednisone (R-CHOP) -all of which are suboptimal. Few patients with double hit lymphoma are cured with this approach. There are retrospective reviews suggesting that a more intensive therapy, such as etoposide, prednisone, vincristine, cyclophosphamide, and doxorubicin plus rituximab (EPOCH-R), may be better than standard treatment. For patients who are unable to undergo transplant, or for those who relapse after a stem cell transplant, the median survival is approximately 6 months.

In view of the foregoing, there remains an unmet need for new drugs that can treat aggressive lymphoma and leukemia subtypes more effectively, particularly forms of lymphoma that are refractory to standard therapy. There also exists a need for new drugs that can treat other hematological cancers that are relatively less known and understood, such as myelodysplastic syndrome and diffuse mixed cell lymphoma. In general, there is a continuing need for anti-cancer drugs with excellent potency, specificity and tolerability.

### SUMMARY

The dihydroorotate dehydrogenase inhibitor 1-methyl-5-(2′-methyl-[1,1′-biphenyl]-yl)-1H-benzo[d][1,2,3]triazole-7-carboxylic acid and its pharmaceutically acceptable salts thereof, also referred to as the “compound of Formula (I)” or “Compound 1” is useful in, among other things, the treatment of hematological malignancies and provides a therapeutic advantage when used in combination with other certain BH3 mimetics, DNA demethylating agents, and standard of care agents, as herein described, compared to treatment with each agent when administered alone.

For example, treatment of acute myeloid leukemia (AML) and diffuse large B-cell lymphoma (DLBCL) cell lines with Compound 1 and the BH3-mimetic venetoclax resulted in synergistic growth inhibition at certain drug concentrations (CI=0.44 in MOLM-13 and CI<0.75 in Pfeiffer; FIG. 1). In the same cell lines, the DNA demethylating agent azacitidine resulted in modest synergy in growth inhibition when combined with Compound 1 in the AML cell line (MOLM-13; CI=0.79), but not the DLBCL cell line (Pfeiffer; CI=0.96; FIG. 2). As shown below in Example 3, the combination Compound 1 and venetoclax in AML and DLBCL-tumour bearing mice resulted in two-fold and three-fold increases in tumour growth inhibition.

Therefore, in one aspect, provided herein are methods for treating a cancer in a subject, comprising administering to the subject an effective amount of a first anti-cancer agent and an effective amount of at least one second anti-cancer agent, wherein the first anti-cancer agent is represented by Formula (I):

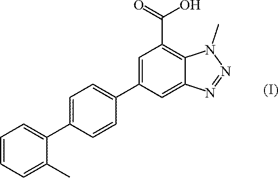 [SMILES: Cc1ccccc1-c1ccc(-c2cc(C(=O)O)c3c(c2)nnn3C)cc1]

or a pharmaceutically acceptable salt thereof; and wherein the at least one second anti-cancer agent is selected from the group consisting of a BH3 mimetic and a DNA demethylating agent, or a combination thereof. In one embodiment, the pharmaceutically acceptable salt thereof is a tris(hydroxymethyl)aminomethane salt or a sodium salt.

In another aspect, this application provides the use of a first anti-cancer agent in the manufacture of a medicament for treating a cancer, wherein the medicament is used in combination with an effective amount of at least one second anti-cancer agent, wherein the first anti-cancer agent is represented by the following structural formula:

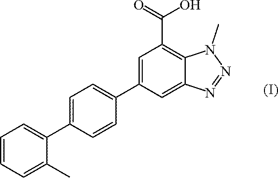 [SMILES: Cc1ccccc1-c1ccc(-c2cc(C(=O)O)c3c(c2)nnn3C)cc1]

or a pharmaceutically acceptable salt thereof; and wherein the at least one second anti-cancer agent is selected from the group consisting of a BH3 mimetic and a DNA demethylating agent, or a combination thereof. In one embodiment, the pharmaceutically acceptable salt thereof is a tris(hydroxymethyl)aminomethane salt or a sodium salt.

In still another aspect, the application provides a first anti-cancer agent for use in treating a cancer in combination with at least one second anti-cancer agent, wherein the first anti-cancer agent is represented by Formula (I):

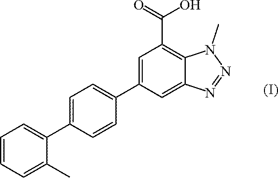 [SMILES: Cc1ccccc1-c1ccc(-c2cc(C(=O)O)c3c(c2)nnn3C)cc1]

or a pharmaceutically acceptable salt thereof; and wherein the at least one second anti-cancer agent is selected from the group consisting of a BH3 mimetic and a DNA demethylating agent, or a combination thereof. In one embodiment, the pharmaceutically acceptable salt thereof is a tris(hydroxymethyl)aminomethane salt or a sodium salt.

In a further aspect, the application provides a pharmaceutical composition comprising a first anti-cancer agent and at least one second anti-cancer agent, wherein the pharmaceutical composition additionally comprises an excipient, wherein the first anti-cancer agent is represented by Formula (I):

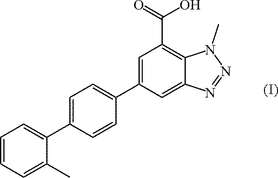 [SMILES: Cc1ccccc1-c1ccc(-c2cc(C(=O)O)c3c(c2)nnn3C)cc1]

or a pharmaceutically acceptable salt thereof; and wherein the at least one second anti-cancer agent is selected from the group consisting of a BH3 mimetic and a DNA demethylating agent, or a combination thereof. In one embodiment, the pharmaceutically acceptable salt thereof is a tris(hydroxymethyl)aminomethane salt or a sodium salt.

Other features or advantages will be apparent from the following detailed description of the drawings and several embodiments, and also from the appended claims.

### BRIEF DESCRIPTION OF THE DRAWINGS

FIG. 1 shows a CTG growth assay on MOLM-13 and Pfeiffer cells treated with varying concentrations of Compound 1 and venetoclax in matrix format for 96 hours. Relative growth rates (compared to DMSO control) for each combination are depicted and color-coded as indicated by the bar to the right of each graph.

FIG. 2 shows a CTG growth assay on MOLM-13 and Pfeiffer cells treated with varying concentrations of Compound 1 and azacitidine in matrix format for 96 hours. Relative growth rates (compared to DMSO control) for each combination are depicted and color-coded as indicated by the bar to the right of each graph.

FIG. 3 shows a CTG growth assay on SUDHL2 cells treated with varying concentrations of Compound 1 and cytarabine in matrix format for 96 hours. Relative growth rates (compared to DMSO control) for each combination are depicted and color-coded as indicated by the bar to the right of each graph.

FIG. 4A shows MOLM13 tumor growth curves in CB17 SCID mice when left untreated (vehicle) or treated with 0.3, 1, or 3 mg/kg of venetoclax, QD.

FIG. 4B shows MOLM13 tumor growth curves in CB17 SCID mice when left untreated (vehicle) or treated with 3, 10, 30, or 100 mg/kg of venetoclax, QD.

FIG. 5 shows average body weight change curves in CB17 SCID mice when treated with varying doses of Compound 1 given BID in combination with 30 mg/k of venetoclax.

FIG. 6A shows MOLM13 tumor growth curves in CB17 SCID mice when left untreated (vehicle) or treated with combinations of Compound 1 (at either 30 mg/kg or 100 mg/kg) and venetoclax (30 mg/kg) using different dosing schedules.

FIG. 6B shows average body weight change curves in CB17 SCID mice when treated with combinations of Compound 1 (either 30 mg/kg or 100 mg/kg) and venetoclax (30 mg/kg) using different dosing schedules.

FIG. 7 shows OCILY19 tumor growth curves in CB17 SCID mice when left untreated (vehicle) or treated with 0.3, 1, or 3 mg/kg of venetoclax, QD.

FIG. 8A shows OCILY19 tumor growth curves in CB17 SCID mice when left untreated (vehicle) or treated with 30 mg/kg of Compound 1, 1 mg/kg venetoclax, or combinations of Compound 1 (either 30 mg/kg or 100 mg/kg) and venetoclax (1 mg/kg) using different dosing schedules.

FIG. 8B shows average body weight change curves in CB17 SCID mice when treated with 30 mg/kg of Compound 1, venetoclax, or combinations of Compound 1 (either 30 mg/kg or 100 mg) and venetoclax (1 mg/kg) using different dosing schedules.

FIG. 9 shows average body weight change curves in CB17 SCID mice when treated with varying doses of Compound 1 given BID or on a holiday schedule in combination with 30 mg/kg of venetoclax and 5 mg/kg of azacitidine.

### DETAILED DESCRIPTION

1-methyl-5-(2′-methyl-[1,1′-biphenyl]-yl)-1H-benzo[d][1,2,3]triazole-7-carboxylic acid, hereinafter also referred to as the “compound of Formula (I)” or “Compound 1” has the chemical structure shown below and is characterized as an inhibitor of DHODH. See e.g., International Patent Application Publication Nos. WO 2014/128669 and WO 2019/164794 and U.S. Pat. No. 9,630,932, the contents of which are incorporated herein by reference.

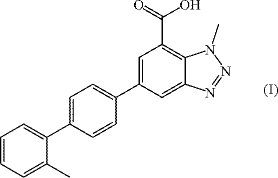 [SMILES: Cc1ccccc1-c1ccc(-c2cc(C(=O)O)c3c(c2)nnn3C)cc1]

The compound of Formula (I) was developed to treat conditions and disorders that would benefit from inhibition of DHODH, such as but not limited to solid cancers and hematological cancers.

In one aspect, the subject application is directed to a method for treating a cancer in a subject by administering an effective amount of the compound of Formula (I) or a pharmaceutically acceptable salt thereof, in combination with an effective amount of a BH3 mimetic and a DNA demethylating agent, or a combination thereof.

In one aspect, the cancer being treated by the disclosed methods is responsive to inhibition of dihydroorotate dehydrogenase.

In one aspect, the cancer being treated by the disclosed methods is a hematological cancer. In some embodiments, the hematological cancer is selected from myeloma, lymphoma, and leukemia. In some embodiments, the hematological cancer is lymphoma or leukemia. In some embodiments, the hematological cancer is selected from acute myeloid leukemia, multiple myeloma, B-prolymphocytic leukemia, acute lymphoblastic leukemia, chronic lymphocytic leukemia, Hodgkin’s disease, non-Hodgkin’s lymphoma, follicular lymphoma, diffuse large B cell lymphoma, anaplastic large cell lymphoma, mantle cell lymphoma, lymphocytic lymphoma cancer of the bladder, and T-cell lymphoma. In one embodiment, the cancer is myelodysplastic/myeloproliferative neoplasms. In some embodiments, the hematological cancer is selected from chemotherapy-resistant acute myeloid leukemia, cytarabine-resistant acute myeloid leukemia, acute monocytic leukemia, acute lymphoblastic leukemia, B-cell lymphoma, diffuse mixed cell lymphoma, myelodysplastic syndrome, primary effusion lymphoma, erythroleukemia, chronic myeloid leukemia, chronic monocytic leukemia, double hit diffuse large B cell lymphoma, and triple hit diffuse large B cell lymphoma. In some embodiments, the hematological cancer is selected from angioimmunoblastic lymphoma, Burkitt’s lymphoma, Burkitt-like lymphoma, blastic NK-cell lymphoma, cutaneous T-cell lymphoma, lymphoblastic lymphoma, MALT lymphoma, mediastinal large B-cell lymphoma, nodal marginal zone B-cell lymphoma, small lymphocytic lymphoma, thyroid lymphoma, follicular lymphoma, Waldenstrom’s macroglobulinemia, essential thrombocythemia, chronic idiopathic myelofibrosis, and polyeythemia rubra vera. In some embodiments, the hematological cancer is acute myeloid leukemia or diffuse large B-cell lymphoma. In another aspect, the cancer being treated by the disclosed methods is a solid tumor. In some embodiments, the solid cancer is selected from lung cancer, breast cancer (e.g., triple negative breast cancer), melanoma, glioblastoma, prostate cancer, colon cancer, pancreatic cancer, bone cancer, cancer of the head or neck, skin cancer, cutaneous or intraocular malignant endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the kidney or ureter, carcinoma of the renal pelvis, neoplasm of the central nervous system (CNS), tumor angiogenesis, spinal axis tumor, brain stem glioma, pituitary adenoma, Kaposi’s sarcoma, epidermoid cancer, squamous cell cancer, an environmentally induced cancer, and a PTEN mutant cancer. In some embodiments, the cancer is selected from biliary tract cancer or cancer of the ampulla of Vater, non-small cell lung cancer, bronchoalveolar carcinoma, liver cancer, cancer of the ovary, cancer of the upper aerodigestive tract, and a cancer induced by asbestos. In some embodiments, the cancer is selected from biliary tract cancer, cancer of the ampulla of Vater, non-small cell lung cancer, bronchoalveolar carcinoma, liver cancer, cancer of the ovary, and cancer of the upper aerodigestive tract. In some embodiments, the cancer is selected from triple negative breast cancer, melanoma, prostate cancer, and cancer of the esophagus. In one embodiment, the cancer is lung cancer. In one embodiment, the cancer is colon cancer.

In one embodiment, the cancer being treated by the disclosed methods is a non-Hodgkin lymphoma or Hodgkin lymphoma. In one particular embodiment, the treated subject belongs to a subpopulation of non-Hodgkin lymphoma or Hodgkin lymphoma patients, where the non-Hodgkin lymphoma or Hodgkin lymphoma has progressed in spite of prior treatment, and for whom additional effective (curative or life-prolonging) standard therapy is not available. In other words, the treated subject belongs to a subpopulation of resistant or refractory non-Hodgkin lymphoma or Hodgkin lymphoma. In one embodiment, the standard curative therapy is high-dose chemotherapy and autologous stem cell transplantation (HD-ASCT). In one embodiment, the subject’s non-Hodgkin lymphoma or Hodgkin lymphoma has relapsed after HD-ASCT. In other words, the treated subject belongs to a subpopulation of relapsed non-Hodgkin lymphoma or Hodgkin lymphoma (e.g., HD-ASCT-relapsed). In another embodiment, the subject is not eligible for HD-ASCT. In another embodiment, the subject has refused HD-ASCT.

In one embodiment, the lymphoma being treated by the disclosed methods is a mature B-cell neoplasm, a mature T- and NK-cell neoplasm, a hodgkin lymphoma, or an Immunodeficiency-associated lymphoproliferative disorder.

In one aspect, the cancer being treated by the disclosed methods is selected from acute myeloid leukemia (e.g., chemotherapy-resistant acute myeloid leukemia, cytarabine-resistant acute myeloid leukemia), multiple myeloma, B-prolymphocytic leukemia, acute lymphoblastic leukemia, chronic lymphocytic leukemia, Hodgkin’s disease, non-Hodgkin’s lymphoma, follicular lymphoma, diffuse large B cell lymphoma (e.g., diffuse large B cell lymphoma), anaplastic large cell lymphoma, mantle cell lymphoma, T-cell lymphoma, acute monocytic leukemia, B-cell lymphoma, diffuse mixed cell lymphoma, myelodysplastic syndrome, primary effusion lymphoma, erythroleukemia, chronic myeloid leukemia, chronic monocytic leukemia, double hit diffuse large B cell lymphoma, triple hit diffuse large B cell lymphoma, and small lymphocytic lymphoma (SLL) in a subject.

In one aspect, the cancer being treated by the disclosed methods is selected from acute myeloid leukemia, multiple myeloma, B-prolymphocytic leukemia, acute lymphoblastic leukemia, chronic lymphocytic leukemia, Hodgkin’s disease, non-Hodgkin’s lymphoma, follicular lymphoma, diffuse large B cell lymphoma, anaplastic large cell lymphoma, mantle cell lymphoma, and T-cell lymphoma.

In one aspect, the cancer being treated by the disclosed methods is selected from acute myeloid leukemia, multiple myeloma, B-prolymphocytic leukemia, non-Hodgkin’s lymphoma, diffuse large B cell lymphoma, anaplastic large cell lymphoma, and mantle cell lymphoma.

In one aspect, the cancer being treated by the disclosed methods is selected from chronic lymphocytic leukemia or small lymphocytic lymphoma.

In one embodiment, the hematologic malignancies to be treated are selected from acute myelogenous leukemia (AML), myelodysplastic syndrome (MDS), chronic myelomonocytic leukemia (CMML), myeloid sarcoma, multiple myeloma, lymphoma (e.g., T-cell lymphoma or B-cell lymphoma), angioimmunoblastic T-cell lymphoma (AITL) or blastic plasmacytoid dendritic cell neoplasm. In some embodiments, the hematological malignancy to be treated is an advanced hematological malignancy. In some embodiments, the hematological malignancy to be treated is newly diagnosed AML. In some embodiments, the hematological malignancy is relapsed and/or refractory AML. In some embodiments, the hematological malignancy to be treated is MDS. In some embodiments, the hematological malignancy to be treated is high risk MDS. In some embodiments, the hematological malignancy to be treated is MDS. In one embodiment, MDS is selected from the following disorders: refractory anemia (RA); RA with ringed sideroblasts (RARS); RA with excess of blasts (RAEB); refractory cytopenia with multilineage dysplasia (RCMD), refractory cytopenia with unilineage dysplasia (RCUD); unclassifiable myelodysplastic syndrome (MDS-U), myelodysplastic syndrome associated with an isolated del(5q) chromosome abnormality, therapy-related myeloid neoplasms and chronic myelomonocytic leukemia (CMML). In one embodiment, MDS is selected from lower-risk MDS and higher-risk MDS. In certain embodiments, the lower-risk MDS and higher-risk MDS, are determined by prognostic systems that are based most commonly on blast percentage, cytogenetic risk groups, and cytopenias, but which may also include age, performance status, transfusion needs, and other clinical (and increasingly molecular) factors.

In certain embodiments, patients with higher-risk MDS fall into International Prognostic Scoring System (IPSS) categories of Intermediate-2 and High groups, corresponding largely to IPSS-R groups Very High, High, and, sometimes, Intermediate, and which often correspond to World Health Organization (WHO) histologic subtypes of refractory anemia with excess blasts (RAEB)-1 and RAEB-2, with an expected median overall survival of <2 years. Patients with high risk MDS (INT-2/High IPSS or High/Very high IPSS-R scores) have a 33% to 45% chance, respectively, of progression to AML and a median survival of around 12 months without intervention (Greenberg et al. Clinical application and proposal for modification of the International Working Group (IWG) response criteria in myelodysplasia. Blood 2006;108(2):419-25 1997). In some embodiments, the hematological malignancy to be treated is high risk MDS.

In one embodiment, the cancer treated in the present methods is chemotherapy-resistant acute myeloid leukemia. In one embodiment, the cancer treated is cytarabine-resistant acute myeloid leukemia. In one embodiment, the cancer treated in the method is acute monocytic leukemia. In one embodiment, the cancer treated in the method is acute lymphoblastic leukemia. In one embodiment, the cancer treated in the method is diffuse mixed cell lymphoma. In one embodiment, the cancer treated in the method is myelodysplastic syndrome. In one embodiment, the cancer treated in the method is primary effusion lymphoma. In one embodiment, the cancer treated in the method is erythroleukemia. In one embodiment, the cancer treated in the method is chronic myeloid leukemia. In one embodiment, the cancer treated in the method is chronic monocytic leukemia. In one embodiment, the cancer treated in the method is B-cell lymphoma.

In one aspect, the cancer to be treated is a double hit diffuse large B cell lymphoma or a triple hit diffuse large B cell lymphoma

As used herein the terms “subject” and “patient” may be used interchangeably, and mean a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

An “effective amount” of the compound of Formula (I), or a pharmaceutically acceptable salt, is an amount sufficient to provide a therapeutic benefit in the treatment of a cancer or to delay or minimize one or more symptoms associated with the condition when combined with a second anti-cancer agent, such as a BH3 mimetic or a DNA demethylating agent, or a combination thereof. An “effective amount” of the second anti-cancer agent, as described herein, is an amount sufficient to provide a therapeutic benefit in the treatment of a condition or to delay or minimize one or more symptoms associated with the condition when combined with the compound of Formula (I), or a pharmaceutically acceptable salt thereof. The terms “therapeutically effective amount” and “effective amount” are used interchangeably. The term “effective amount” can encompass an amount that improves overall therapy, reduces or avoids symptoms, signs, or causes of the condition, and/or enhances the therapeutic efficacy of another therapeutic agent. In certain embodiments, an effective amount is an amount sufficient for eliciting therapeutic effects in the treatment of a cancer (including solid tumors and hematological cancers as further described herein).

The precise amount of the compound of Formula (I) (or pharmaceutically acceptable salt thereof), a BH3 mimetic or a DNA demethylating agent, or combinations thereof administered to a subject will depend on various factors, such as the given drug, the pharmaceutical formulation, the route of administration, the type of disease or disorder, the identity of the subject being treated, and the like, but can nevertheless be routinely determined by one skilled in the art. For example, determination of an effective amount will also depend on the degree, severity and type of cell proliferation. The skilled artisan will be able to determine appropriate dosages depending on these and other factors. When co-administered with other therapeutic agents, e.g., when co-administered with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of the compound of Formula (I) or pharmaceutically acceptable salt thereof. In cases where no amount is expressly noted, an effective amount should be assumed. Non-limiting examples of an effective amount of the compound of Formula (I) or pharmaceutically acceptable salt thereof are provided herein below.

An effective amount of the compound of Formula (I), a pharmaceutically acceptable salt thereof, or a second anti-cancer agent is generally in the range from 0.1 µg to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as an individual dose per day or usually in a series of part-doses (such as, for example, two, three, four, five or six) per day, so that the total daily dose is the same. The effective dose of the compound of Formula (I) or pharmaceutically acceptable salt thereof to a subject can be 10 µg -500 mg. Effective amounts of BH3 mimetics and DNA demethylating agents are known to those skilled in the art. Exemplary amounts are further discussed below.

The term “treat” means decrease, suppress, attenuate, diminish, arrest, or stabilize the development or progression of a disease/disorder (e.g., an advanced hematologic malignancy, such as acute myelogenous leukemia (AML), myelodysplastic syndrome (MDS), chronic myelomonocytic leukemia (CMML), myeloid sarcoma, multiple myeloma, or lymphoma (e.g., T-cell lymphoma)), lessen the severity of the disease/disorder or improve the symptoms associated with the disease/disorder. Treatment may also be continued after symptoms have resolved, for example to reduce the likelihood of or delay their recurrence.

Positive therapeutic effects in cancer can be measured in a number of ways. The administration of a therapeutically effective amount of the combinations herein described are advantageous over the individual component compounds. As used herein “advantageous combinations” are those combinations that provide at least one of the following improved properties when compared to the individual administration of a therapeutically effective amount of a component compound: i) a greater anticancer effect than the most active single agent, alone; ii) synergistic anticancer effect; or iii) additive activity.

The term “myelodysplastic syndrome” refers to hematological conditions characterized by abnormalities in the production of one or more of the cellular components of blood (red cells, white cells (other than lymphocytes) and platelets (or their progenitor cells, megakaryocytes)).

The term “relapsed” refers to a situation where, after therapy, patients who have had a remission of cancer, including AML, have a return of cancer cells.

The term “refractory or resistant” refers to a circumstance where patients, even after intensive treatment, have residual cancer cells in their body.

As used herein, “chemotherapy-resistant acute myeloid leukemia” refers to a form of acute myeloid leukemia that is resistant or refractory to standard chemotherapy for acute myeloid leukemia. In one embodiment, the standard chemotherapy for acute myeloid leukemia comprises one or more approved chemotherapeutic agents selected from cytarabine, doxorubicin, daunorubicin (daunomycin), idarubicin, clardribine (Leustatin®, 2-CdA), fludarabine (Fludara®), topotecan, etoposide (VP-16), 6-thioguanine or 6-TG, hydroxyurea (Hydrea®), corticosteroids (e.g., prednisone or dexamethasone (Decadron®), methotrexate or MTX, 6-mercaptopurine or 6-MP, azacitidine (Vidaza®), decitabine (Dacogen®).

As used herein, “cytarabine-resistant acute myeloid leukemia” refers to a form of acute myeloid leukemia that is resistant or refractory to treatment of the disease with cytarabine, alone or in combination with one or more additional therapeutic agents.

As used herein, “double hit diffuse large B cell lymphoma” refers to a form of lymphoma or diffuse large B cell lymphoma where the lymphoma cells are altered at two oncogenes which are c-MYC and BCL2 or BCL6. In one embodiment, double hit diffuse large B cell lymphoma is treated in the method, and is characterized by gene alterations at c-MYC and BCL2. In another embodiment, double hit diffuse large B cell lymphoma is treated in the method, and is characterized by gene alterations at c-MYC and BCL6.

As used herein, “triple hit diffuse large B cell lymphoma” refers to a form of lymphoma or diffuse large B cell lymphoma where the lymphoma cells are altered at three oncogenes which are c-MYC, BCL2 and BCL6. In one embodiment, triple hit diffuse large B cell lymphoma is treated in the method.

In this description, a “pharmaceutically acceptable salt” is a pharmaceutically acceptable, organic or inorganic acid or base salt of a compound described herein. Representative pharmaceutically acceptable, organic or inorganic base salts include, e.g., alkali metal salts (e.g., sodium), alkali earth salts (e.g., calcium), and ammonium salts (e.g., tris(hydroxymethyl)aminomethane, hydrabamine, and N-methylglucamine ammonium salt). Representative pharmaceutically acceptable, organic or inorganic acid or base salts include, e.g., the acetate, amsonate (4,4-diaminostilbene-2, 2 -disulfonate), benzenesulfonate, benzonate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

In one embodiment, the methods provided herein comprise administration or co-administration of one or more BH3 mimetics. BH3 mimetics are compounds that target, decrease, or inhibit antiapoptotic proteins from the BCL-2 family. Exemplary nonlimiting BH3 mimetics include venetoclax, BGB-11417, navitoclax (ABT-263), LP-108, S55746, S65487, AT-101, APG-1252, obatoclax, APG2575, and BCL-201.

The term “co-administering” as used herein with respect to the at least one second anti-cancer agent means that the at least one second anti-cancer agent may be administered together with Compound 1, or a pharmaceutically acceptable salt thereof, as part of a single dosage form (such as a composition comprising Compound 1, or a pharmaceutically acceptable salt thereof, and the at least one second anti-cancer agent) or as separate, multiple dosage forms. Alternatively, the at least one second anti-cancer agent may be administered prior to, consecutively with, or following the administration of Compound 1, or a pharmaceutically acceptable salt thereof. In such combination therapy treatment, both Compound 1, or a pharmaceutically acceptable salt thereof, and the at least one second anti-cancer agent are administered by conventional methods. The administration of a composition comprising both Compound 1, or a pharmaceutically acceptable salt thereof, and the at least one second anti-cancer agent, to a subject does not preclude the separate administration of that same therapeutic agent, any other second therapeutic agent or any compound provided herein to said subject at another time during a course of treatment. The term “co-administering” as used herein with respect to an additional cancer treatment means that the additional cancer treatment may occur prior to, consecutively with, concurrently with or following the administration of a compound provided herein.

In one embodiment, the BH3 mimetic used the disclosed methods and compositions is venetoclax.

Venetoclax (VENCLEXTA®) is approved for the treatment (1) of adult patients with chronic lymphocytic leukemia (CLL) or small lymphocytic lymphoma (SLL); and (2) in combination with azacitidine or decitabine or low-dose cytarabine for the treatment of newly-diagnosed acute myeloid leukemia (AML) in adults who are age 75 years or older, or who have comorbidities that preclude use of intensive induction chemotherapy. It is supplied as tablets for oral administration that contain 10, 50, or 100 mg venetoclax as the active ingredient.

Venetoclax is a selective inhibitor of BCL-2 protein. It is a light yellow to dark yellow solid with the empirical formula C₄₅H₅₀ClN₇O₇S and a molecular weight of 868.44. Venetoclax is described chemically as 4-(4-{ [2-(4-chlorophenyl)-4,4- dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide) and has the following chemical structure:

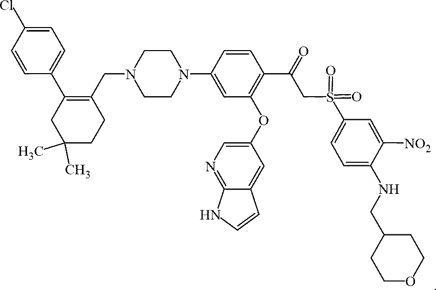

In one embodiment, depending on the disease to be treated and the subject’s condition, venetoclax may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. Venetoclax may be formulated, alone or together with Compound 1 and/or one or more active agent(s), in suitable dosage unit with pharmaceutically acceptable excipients, carriers, adjuvants and vehicles, appropriate for each route of administration.

In one embodiment, venetoclax is administered by, e.g., intravenous (IV), subcutaneous (SC) or oral routes. Certain embodiments herein provide co-administration of venetoclax with Compound 1, or a pharmaceutically acceptable salt thereof, and/or one or more additional active agents to provide a therapeutic advantage in subjects in need thereof. Certain embodiments herein provide co-administration of venetoclax with Compound 1, or a pharmaceutically acceptable salt thereof, and/or one or more additional active agents to provide a synergistic therapeutic effect in subjects in need thereof. The co-administered active agent(s) may be additional cancer therapeutic agents, as described herein (e.g., other DNA demethylating agents, BH3 mimetics, or standards of care). In certain embodiments, the co-administered active agent(s) is azacitidine. In certain embodiments, the co-administered agent(s) may be dosed, e.g., orally or by injection (e.g., IV or SC).

In certain embodiments, treatment cycles comprise multiple doses of venetoclax administered to a subject in need thereof over multiple days (e.g., 1, 2, 3, 4, 5, 6, 7, 8, 9, 10, 11, 12, 13, 14, or greater than 14 days). In certain embodiments, treatment cycles comprise multiple doses of venetoclax administered to a subject in need thereof over multiple weeks (e.g., 1, 2, 3, 4, or greater than 5 weeks). Suitable dosage amounts for the methods provided herein include, e.g., therapeutically effective amounts and prophylactically effective amounts. For example, in certain embodiments, the amount of venetoclax administered in the methods provided herein may range, e.g., between about 5 mg/day and about 2,000 mg/day. In one embodiment, the range is between about 10 mg/day and about 2,000 mg/day. In one embodiment, the range is between about 20 mg/day and about 2,000 mg/day. In one embodiment, the range is between about 50 mg/day and about 1,000 mg/day. In one embodiment, the range is between about 100 mg/day and about 1,000 mg/day. In one embodiment, the range is between about 100 mg/day and about 500 mg/day. In one embodiment, the range is between about 150 mg/day and about 500 mg/day. In one embodiment, the range is between about 150 mg/day and about 250 mg/day. In one embodiment, the range is between about 15 mg/day and about 425 mg/day. In certain embodiments, the particular dosage is about 10 mg/day. In one embodiment, the particular dosage is about 20 mg/day. In one embodiment, the particular dosage is about 50 mg/day. In one embodiment, the particular dosage is about 75 mg/day. In one embodiment, the particular dosage is about 100 mg/day. In one embodiment, the particular dosage is about 120 mg/day. In one embodiment, the particular dosage is about 150 mg/day. In one embodiment, the particular dosage is about 200 mg/day. In one embodiment, the particular dosage is about 250 mg/day. In one embodiment, the particular dosage is about 300 mg/day. In one embodiment, the particular dosage is about 350 mg/day. In one embodiment, the particular dosage is about 400 mg/day. In one embodiment, the particular dosage is about 450 mg/day. In one embodiment, the particular dosage is about 500 mg/day. In one embodiment, the particular dosage is about 600 mg/day. In one embodiment, the particular dosage is about 700 mg/day. In one embodiment, the particular dosage is about 800 mg/day. In one embodiment, the particular dosage is about 900 mg/day. In one embodiment, the particular dosage is about 1,000 mg/day. In one embodiment, the particular dosage is about 1,200 mg/day. In one embodiment, the particular dosage is about 1,500 mg/day. In certain embodiments, the particular dosage is up to about 10 mg/day. In one embodiment, the particular dosage is up to about 20 mg/day. In one embodiment, the particular dosage is up to about 50 mg/day. In one embodiment, the particular dosage is up to about 75 mg/day. In one embodiment, the particular dosage is up to about 100 mg/day. In one embodiment, the particular dosage is up to about 120 mg/day. In one embodiment, the particular dosage is up to about 150 mg/day. In one embodiment, the particular dosage is up to about 200 mg/day. In one embodiment, the particular dosage is up to about 250 mg/day. In one embodiment, the particular dosage is up to about 300 mg/day. In one embodiment, the particular dosage is up to about 350 mg/day. In one embodiment, the particular dosage is up to about 400 mg/day. In one embodiment, the particular dosage is up to about 450 mg/day. In one embodiment, the particular dosage is up to about 500 mg/day. In one embodiment, the particular dosage is up to about 600 mg/day. In one embodiment, the particular dosage is up to about 700 mg/day. In one embodiment, the particular dosage is up to about 800 mg/day. In one embodiment, the particular dosage is up to about 900 mg/day. In one embodiment, the particular dosage is up to about 1,000 mg/day. In one embodiment, the particular dosage is up to about 1,200 mg/day. In one embodiment, the particular dosage is up to about 1,500 mg/day.

In one embodiment, the amount of venetoclax in the pharmaceutical composition or dosage form provided herein may range, e.g., between about 5 mg and about 2,000 mg. In one embodiment, the range is between about 10 mg and about 2,000 mg. In one embodiment, the range is between about 20 mg and about 2,000 mg. In one embodiment, the range is between about 50 mg and about 1,000 mg. In one embodiment, the range is between about 50 mg and about 500 mg. In one embodiment, the range is between about 50 mg and about 250 mg. In one embodiment, the range is between about 100 mg and about 500 mg. In one embodiment, the range is between about 150 mg and about 500 mg. In one embodiment, the range is between about 150 mg and about 250 mg. In one embodiment, the range is between about 15 mg/day and about 425 mg/day. In certain embodiments, the particular amount is about 10 mg. In one embodiment, the particular amount is about 20 mg. In one embodiment, the particular amount is about 50 mg. In one embodiment, the particular amount is about 75 mg. In one embodiment, the particular amount is about 100 mg. In one embodiment, the particular amount is about 120 mg. In one embodiment, the particular amount is about 150 mg. In one embodiment, the particular amount is about 200 mg. In one embodiment, the particular amount is about 250 mg. In one embodiment, the particular amount is about 300 mg. In one embodiment, the particular amount is about 350 mg. In one embodiment, the particular amount is about 400 mg. In one embodiment, the particular amount is about 450 mg. In one embodiment, the particular amount is about 500 mg. In one embodiment, the particular amount is about 600 mg. In one embodiment, the particular amount is about 700 mg. In one embodiment, the particular amount is about 800 mg. In one embodiment, the particular amount is about 900 mg. In one embodiment, the particular amount is about 1,000 mg. In one embodiment, the particular amount is about 1,200 mg. In one embodiment, the particular amount is about 1,500 mg. In certain embodiments, the particular amount is up to about 10 mg. In one embodiment, the particular amount is up to about 20 mg. In one embodiment, the particular amount is up to about 50 mg. In one embodiment, the particular amount is up to about 75 mg. In one embodiment, the particular amount is up to about 100 mg. In one embodiment, the particular amount is up to about 120 mg. In one embodiment, the particular amount is up to about 150 mg. In one embodiment, the particular amount is up to about 200 mg. In one embodiment, the particular amount is up to about 250 mg. In one embodiment, the particular amount is up to about 300 mg. In one embodiment, the particular amount is up to about 350 mg. In one embodiment, the particular amount is up to about 400 mg. In one embodiment, the particular amount is up to about 450 mg. In one embodiment, the particular amount is up to about 500 mg. In one embodiment, the particular amount is up to about 600 mg. In one embodiment, the particular amount is up to about 700 mg. In one embodiment, the particular amount is up to about 800 mg. In one embodiment, the particular amount is up to about 900 mg. In one embodiment, the particular amount is up to about 1,000 mg. In one embodiment, the particular amount is up to about 1,200 mg. In one embodiment, the particular amount is up to about 1,500 mg.

In one embodiment, venetoclax can be delivered as a single dose such as, e.g., a single bolus injection, or oral tablets or pills; or over time such as, e.g., continuous infusion over time or divided bolus doses over time. In one embodiment, venetoclax can be administered repetitively if necessary, for example, until the patient experiences stable disease or regression, or until the patient experiences disease progression or unacceptable toxicity. Stable disease or lack thereof is determined by methods known in the art such as evaluation of patient’s symptoms, physical examination, visualization of the tumor that has been imaged using X-ray, CAT, PET, or MRI scan and other commonly accepted evaluation modalities.

In one embodiment, venetoclax can be administered once daily or divided into multiple daily doses such as twice daily, three times daily, and four times daily. In one embodiment, the administration can be continuous (i.e., daily for consecutive days or every day), intermittent, e.g., in cycles (i.e., including days, weeks, or months of rest when no drug is administered). In one embodiment, venetoclax is administered daily, for example, once or more than once each day for a period of time. In one embodiment, venetoclax is administered daily for an uninterrupted period of at least 7 days. In some embodiments, venetoclax is administered for periods lasting longer than 5 weeks. In one embodiment, venetoclax is administered intermittently, i.e., stopping and starting at either regular or irregular intervals. In one embodiment, venetoclax is administered for one to six days per week. In one embodiment, venetoclax is administered on alternate days. In one embodiment, venetoclax is administered in cycles (e.g., administered daily or continuously for a certain period interrupted with a rest period). In one embodiment, venetoclax is administered daily for two to eight consecutive weeks, then a rest period with no administration for up to one week; or e.g., daily administration for one week, then a rest period with no administration for up to three weeks).

In one embodiment, the frequency of administration ranges from about daily to about monthly. In one embodiment, venetoclax is administered once a day. In another embodiment, venetoclax is administered twice a day. In yet another embodiment, venetoclax is administered three times a day. In still another embodiment, venetoclax is administered four times a day. In one embodiment, venetoclax is administered once every other day. In one embodiment, venetoclax is administered twice a week. In one embodiment, venetoclax is administered once every week. In one embodiment, venetoclax is administered once every two weeks. In one embodiment, venetoclax is administered once every three weeks. In one embodiment, venetoclax is administered once every four weeks.

In one embodiment, venetoclax is administered orally for 7 days, followed by a ramp up dosing schedule to a recommended daily dose. In one embodiment, 20 mg/day of venetoclax is administered on days 1-7. In one embodiment, the recommended daily dose is 400 mg. In one embodiment, venetoclax is administered using the following protocol:

| Week | Dosage |
|---|---|
| 1 | 20 mg |
| 2 | 50 mg |
| 3 | 100 mg |
| 4 | 200 mg |
| 5 | 400 mg |

In one embodiment, the methods provided herein comprise administration or co-administration of one or more DNA demethylating agents.

The term “DNA demethylating agent” refers to an agent that inhibits the transfer of a methyl group to DNA. In one embodiment, the DNA demethylating agent is a cytidine analog.

The term “a cytidine analog” referred to herein is intended to encompass the free base of the cytidine analog, or a salt, solvate, hydrate, cocrystal, complex, prodrug, precursor, metabolite, and/or derivative thereof. In certain embodiments, a cytidine analog referred to herein encompasses the free base of the cytidine analog, or a salt, solvate, hydrate, cocrystal or complex thereof. In certain embodiments, a cytidine analog referred to herein encompasses the free base of the cytidine analog, or a pharmaceutically acceptable salt, solvate, or hydrate thereof.

In certain embodiments, the cytidine analog is 5-azacitidine (azacytidine, azacytidine) or 5-aza-2′-deoxycytidine (decitabine). In certain embodiments, the cytidine analog is, for example: 1-β-D-arabinofuranosylcytosine (Cytarabine or ara-C); pseudoiso-cytidine (psi ICR); 5-fluoro-2′-deoxycytidine (FCdR); 2′-deoxy-2′,2′-difluorocytidine (Gemcitabine); 5-aza-2′-deoxy-2′,2′-difluorocytidine; 5-aza-2′-deoxy-2′-fluorocytidine; 1-β-D-ribofuranosyl-2(1H)-pyrimidinone (Zebularine); 2′,3′-dideoxy-5-fluoro-3′-thiacytidine (Emtriva); 2′-cyclocytidine (Ancitabine); 1-β-D-arabinofuranosyl-5-azacytosine (Fazarabine or ara-AC); 6-azacitidine (6-aza-CR); 5,6-dihydro-5-azacitidine (dH-aza-CR); N⁴-pentyloxy-carbonyl-5′-deoxy-5-fluorocytidine (Capecitabine); N⁴-octadecyl-cytarabine; or elaidic acid cytarabine. In certain embodiments, the cytidine analogs include any compound which is structurally related to cytidine or deoxycytidine and functionally mimics and/or antagonizes the action of cytidine or deoxycytidine.

Exemplary cytidine analogs have the structures provided below:

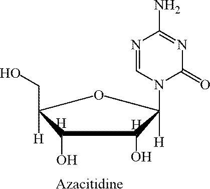

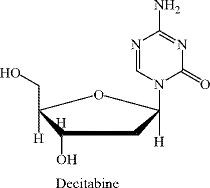

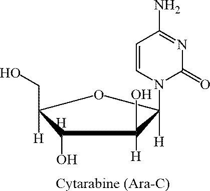

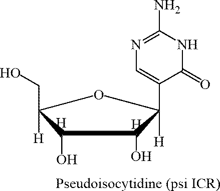

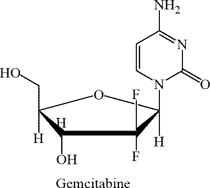

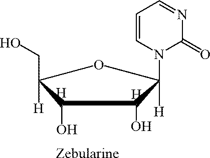

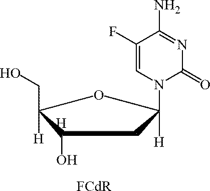

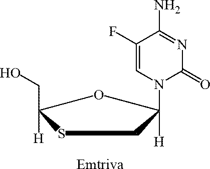

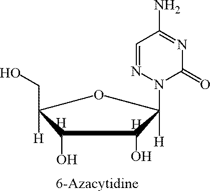

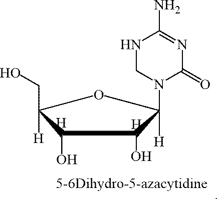

In certain embodiments, the cytidine analog is azacitidine.

Azacitidine is 4-amino-1-β-D-ribofuranozyl-s-triazin-2(1H)-one, also known as VIDAZA® (Celgene Corporation). Its empirical formula is C₈H₁₂N₄O₅, the molecular weight is 244. Azacitidine is a white to off-white solid that is insoluble in acetone, ethanol and methyl ketone; slightly soluble in ethanol/water (50/50), propylene glycol and polyethylene glycol; sparingly soluble in water, water-saturated octanol, 5% dextrose in water, N-methyl-2-pyrrolidone, normal saline and 5% Tween 80 in water, and soluble in dimethylsulfoxide (DMSO).

VIDAZA® is approved for treatment in patients with higher-risk MDS. It is supplied in a sterile form for reconstitution as a suspension for subcutaneous injection or reconstitution as a solution with further dilution for intravenous infusion. Vials of VIDAZA® contain 100 mg of azacitidine and 100 mg of mannitol as a sterile lyophilized powder. The approved dosing schedule is a twice-daily subcutaneous injection or a single daily intravenous infusion on seven consecutive days of a 28-day treatment cycle.

Oral azacitidine is effective and safe in lower-risk myelodisplastic syndrome (MDS) and acute myeloid leukemia (AML) patients. In one embodiment, the dose used in MDS and AML patients is 300 mg once daily based on extended dosing (14 or 21 days of the 28-day treatment cycle). In one embodiment, the starting dose for oral azacitidine is 120 mg and the maximum tolerated dose is 480 mg.

In one embodiment, depending on the disease to be treated and the subject’s condition, azacitidine may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. Azacitidine may be formulated, alone or together with Compound 1 and/or one or more active agent(s), in suitable dosage unit with pharmaceutically acceptable excipients, carriers, adjuvants and vehicles, appropriate for each route of administration.

In one embodiment, azacitidine is administered by, e.g., intravenous (IV), subcutaneous (SC) or oral routes. Certain embodiments herein provide co-administration of azacitidine with Compound 1, or a pharmaceutically acceptable salt thereof, and/or one or more additional active agents to provide a therapeutic advantage in subjects in need thereof. Certain embodiments herein provide co-administration of azacitidine with Compound 1, or a pharmaceutically acceptable salt thereof, and/or one or more additional active agents to provide a synergistic therapeutic effect in subjects in need thereof. The co-administered active agent(s) may be additional cancer therapeutic agents, as described herein (e.g., other DNA demethylating agents, BH3 mimetics, or standards of care). In certain embodiments, the co-administered active agent(s) is venetoclax. In certain embodiments, the co-administered agent(s) may be dosed, e.g., orally or by injection (e.g., IV or SC).

In certain embodiments, treatment cycles comprise multiple doses of azacitidine administered to a subject in need thereof over multiple days (e.g., 1, 2, 3, 4, 5, 6, 7, 8, 9, 10, 11, 12, 13, 14, or greater than 14 days), optionally followed by treatment dosing holidays (e.g., 1, 2, 3, 4, 5, 6, 7, 8, 9, 10, 11, 12, 13, 14, 15, 16, 17, 18, 19, 20, 21, 22, 23, 24, 25, 26, 27, 28, or greater than 28 days). Suitable dosage amounts for the methods provided herein include, e.g., therapeutically effective amounts and prophylactically effective amounts. For example, in certain embodiments, the amount of azacitidine administered in the methods provided herein may range, e.g., between about 50 mg/m²/day and about 2,000 mg/m²/day. In certain embodiments, the amount of azacitidine is between about 100 mg/m²/day and about 1,000 mg/m²/day. In certain embodiments, the amount of azacitidine is between about 100 mg/m²/day and about 500 mg/m²/day. In certain embodiments, the amount of azacitidine is between about 50 mg/m²/day and about 500 mg/m²/day. In certain embodiments, the amount of azacitidine is between about 50 mg/m²/day and about 200 mg/m²/day. In certain embodiments, the amount of azacitidine is between about 50 mg/m²/day and about 100 mg/m²/day. In certain embodiments, the amount of azacitidine is between about 50 mg/m²/day and about 75 mg/m²/day. In certain embodiments, the amount of azacitidine is between about 120 mg/m²/day and about 250 mg/m²/day. In certain embodiments, the particular dosage is about 50 mg/m²/day. In one embodiment, the particular dosage is about 60 mg/m²/day. In one embodiment, the particular dosage is about 75 mg/m²/day. In one embodiment, the particular dosage is about 80 mg/m²/day. In one embodiment, the particular dosage is about 100 mg/m²/day. In one embodiment, the particular dosage is about 120 mg/m²/day. In one embodiment, the particular dosage is about 140 mg/m²/day. In one embodiment, the particular dosage is about 150 mg/m²/day. In one embodiment, the particular dosage is about 180 mg/m²/day. In one embodiment, the particular dosage is about 200 mg/m²/day. In one embodiment, the particular dosage is about 220 mg/m²/day. In one embodiment, the particular dosage is about 240 mg/m²/day. In one embodiment, the particular dosage is about 250 mg/m²/day. In one embodiment, the particular dosage is about 260 mg/m²/day. In one embodiment, the particular dosage is about 280 mg/m²/day. In one embodiment, the particular dosage is about 300 mg/ m²/day. In one embodiment, the particular dosage is about 320 mg/m²/day. In one embodiment, the particular dosage is about 350 mg/m²/day. In one embodiment, the particular dosage is about 380 mg/m²/day. In one embodiment, the particular dosage is about 400 mg/m²/day. In one embodiment, the particular dosage is about 450 mg/m²/day. In one embodiment, the particular dosage is about 500 mg/m²/day. In certain embodiments, the particular dosage is up to about 100 mg/m²/day. In one embodiment, the particular dosage is up to about 120 mg/m²/day. In one embodiment, the particular dosage is up to about 140 mg/m²/day. In one embodiment, the particular dosage is up to about 150 mg/m²/day. In one embodiment, the particular dosage is up to about 180 mg/m²/day. In one embodiment, the particular dosage is up to about 200 mg/m²/day. In one embodiment, the particular dosage is up to about 220 mg/m²/day. In one embodiment, the particular dosage is up to about 240 mg/m²/day. In one embodiment, the particular dosage is up to about 250 mg/m²/day. In one embodiment, the particular dosage is up to about 260 mg/m²/day. In one embodiment, the particular dosage is up to about 280 mg/m²/day. In one embodiment, the particular dosage is up to about 300 mg/ m²/day. In one embodiment, the particular dosage is up to about 320 mg/m²/day. In one embodiment, the particular dosage is up to about 350 mg/m²/day. In one embodiment, the particular dosage is up to about 380 mg/m²/day. In one embodiment, the particular dosage is up to about 400 mg/m²/day. In one embodiment, the particular dosage is up to about 450 mg/m²/day. In one embodiment, the particular dosage is up to about 500 mg/m²/day. In one embodiment, the particular dosage is up to about 750 mg/m²/day. In one embodiment, the particular dosage is up to about 1000 mg/m²/day.

In one embodiment, the amount of azacitidine administered in the methods provided herein may range, e.g., between about 5 mg/day and about 2,000 mg/day. In one embodiment, the range is between about 10 mg/day and about 2,000 mg/day. In one embodiment, the range is between about 20 mg/day and about 2,000 mg/day. In one embodiment, the range is between about 50 mg/day and about 1,000 mg/day. In one embodiment, the range is between about 100 mg/day and about 1,000 mg/day. In one embodiment, the range is between about 100 mg/day and about 500 mg/day. In one embodiment, the range is between about 150 mg/day and about 500 mg/day. In one embodiment, the range is between about 150 mg/day and about 250 mg/day. In certain embodiments, the particular dosage is about 10 mg/day. In one embodiment, the particular dosage is about 20 mg/day. In one embodiment, the particular dosage is about 50 mg/day. In one embodiment, the particular dosage is about 75 mg/day. In one embodiment, the particular dosage is about 100 mg/day. In one embodiment, the particular dosage is about 120 mg/day. In one embodiment, the particular dosage is about 150 mg/day. In one embodiment, the particular dosage is about 200 mg/day. In one embodiment, the particular dosage is about 250 mg/day. In one embodiment, the particular dosage is about 300 mg/day. In one embodiment, the particular dosage is about 350 mg/day. In one embodiment, the particular dosage is about 400 mg/day. In one embodiment, the particular dosage is about 450 mg/day. In one embodiment, the particular dosage is about 500 mg/day. In one embodiment, the particular dosage is about 600 mg/day. In one embodiment, the particular dosage is about 700 mg/day. In one embodiment, the particular dosage is about 800 mg/day. In one embodiment, the particular dosage is about 900 mg/day. In one embodiment, the particular dosage is about 1,000 mg/day. In one embodiment, the particular dosage is about 1,200 mg/day. In one embodiment, the particular dosage is about 1,500 mg/day. In certain embodiments, the particular dosage is up to about 10 mg/day. In one embodiment, the particular dosage is up to about 20 mg/day. In one embodiment, the particular dosage is up to about 50 mg/day. In one embodiment, the particular dosage is up to about 75 mg/day. In one embodiment, the particular dosage is up to about 100 mg/day. In one embodiment, the particular dosage is up to about 120 mg/day. In one embodiment, the particular dosage is up to about 150 mg/day. In one embodiment, the particular dosage is up to about 200 mg/day. In one embodiment, the particular dosage is up to about 250 mg/day. In one embodiment, the particular dosage is up to about 300 mg/day. In one embodiment, the particular dosage is up to about 350 mg/day. In one embodiment, the particular dosage is up to about 400 mg/day. In one embodiment, the particular dosage is up to about 450 mg/day. In one embodiment, the particular dosage is up to about 500 mg/day. In one embodiment, the particular dosage is up to about 600 mg/day. In one embodiment, the particular dosage is up to about 700 mg/day. In one embodiment, the particular dosage is up to about 800 mg/day. In one embodiment, the particular dosage is up to about 900 mg/day. In one embodiment, the particular dosage is up to about 1,000 mg/day. In one embodiment, the particular dosage is up to about 1,200 mg/day. In one embodiment, the particular dosage is up to about 1,500 mg/day.

In one embodiment, the amount of azacitidine in the pharmaceutical composition ordosage form provided herein may range, e.g., between about 5 mg and about 2,000 mg. In one embodiment, the range is between about 10 mg and about 2,000 mg. In one embodiment, the range is between about 20 mg and about 2,000 mg. In one embodiment, the range is between about 50 mg and about 1,000 mg. In one embodiment, the range is between about 50 mg and about 500 mg. In one embodiment, the range is between about 50 mg and about 250 mg. In one embodiment, the range is between about 100 mg and about 500 mg. In one embodiment, the range is between about 150 mg and about 500 mg. In one embodiment, the range is between about 150 mg and about 250 mg. In certain embodiments, the particular amount is about 10 mg. In one embodiment, the particular amount is about 20 mg. In one embodiment, the particular amount is about 50 mg. In one embodiment, the particular amount is about 75 mg. In one embodiment, the particular amount is about 100 mg. In one embodiment, the particular amount is about 120 mg. In one embodiment, the particular amount is about 150 mg. In one embodiment, the particular amount is about 200 mg. In one embodiment, the particular amount is about 250 mg. In one embodiment, the particular amount is about 300 mg. In one embodiment, the particular amount is about 350 mg. In one embodiment, the particular amount is about 400 mg. In one embodiment, the particular amount is about 450 mg. In one embodiment, the particular amount is about 500 mg. In one embodiment, the particular amount is about 600 mg. In one embodiment, the particular amount is about 700 mg. In one embodiment, the particular amount is about 800 mg. In one embodiment, the particular amount is about 900 mg. In one embodiment, the particular amount is about 1,000 mg. In one embodiment, the particular amount is about 1,200 mg. In one embodiment, the particular amount is about 1,500 mg. In certain embodiments, the particular amount is up to about 10 mg. In one embodiment, the particular amount is up to about 20 mg. In one embodiment, the particular amount is up to about 50 mg. In one embodiment, the particular amount is up to about 75 mg. In one embodiment, the particular amount is up to about 100 mg. In one embodiment, the particular amount is up to about 120 mg. In one embodiment, the particular amount is up to about 150 mg. In one embodiment, the particular amount is up to about 200 mg. In one embodiment, the particular amount is up to about 250 mg. In one embodiment, the particular amount is up to about 300 mg. In one embodiment, the particular amount is up to about 350 mg. In one embodiment, the particular amount is up to about 400 mg. In one embodiment, the particular amount is up to about 450 mg. In one embodiment, the particular amount is up to about 500 mg. In one embodiment, the particular amount is up to about 600 mg. In one embodiment, the particular amount is up to about 700 mg. In one embodiment, the particular amount is up to about 800 mg. In one embodiment, the particular amount is up to about 900 mg. In one embodiment, the particular amount is up to about 1,000 mg. In one embodiment, the particular amount is up to about 1,200 mg. In one embodiment, the particular amount is up to about 1,500 mg.

In one embodiment, azacitidine can be delivered as a single dose such as, e.g., a single bolus injection, or oral tablets or pills; or over time such as, e.g., continuous infusion over time or divided bolus doses over time. In one embodiment, azacitidine can be administered repetitively if necessary, for example, until the patient experiences stable disease or regression, or until the patient experiences disease progression or unacceptable toxicity. Stable disease or lack thereof is determined by methods known in the art such as evaluation of patient’s symptoms, physical examination, visualization of the tumor that has been imaged using X-ray, CAT, PET, or MRI scan and other commonly accepted evaluation modalities.

In one embodiment, azacitidine can be administered once daily or divided into multiple daily doses such as twice daily, three times daily, and four times daily. In one embodiment, the administration can be continuous (i.e., daily for consecutive days or every day), intermittent, e.g., in cycles (i.e., administered daily or continuously for a certain period interrupted with a rest period where not drug is administered). In one embodiment, azacitidine is administered daily, for example, once or more than once each day for a period of time. In one embodiment, azacitidine is administered daily for an uninterrupted period of at least 7 days. In some embodiments, azacitidine is administered up to 52 weeks. In one embodiment, azacitidine is administered intermittently, i.e., stopping and starting at either regular or irregular intervals. In one embodiment, azacitidine is administered for one to six days per week. In one embodiment, azacitidine is administered on alternate days. In one embodiment, azacitidine is administered daily for two to eight consecutive weeks, then a rest period with no administration for up to one week; or e.g., daily administration for one week, then a rest period with no administration for up to three weeks).

In one embodiment, the frequency of administration ranges from about daily to about monthly. In one embodiment, azacitidine is administered once a day. In another embodiment, azacitidine is administered twice a day. In yet another embodiment, azacitidine is administered three times a day. In still another embodiment, azacitidine is administered four times a day. In one embodiment, azacitidine is administered once every other day. In one embodiment, azacitidine is administered twice a week. In one embodiment, azacitidine is administered once every week. In one embodiment, azacitidine is administered once every two weeks. In one embodiment, azacitidine is administered once every three weeks. In one embodiment, azacitidine is administered once every four weeks.

In one embodiment, azacitidine is administered once per day from one day to six months. In one embodiment, azacitidine is administered from one week to three months. In one embodiment, azacitidine is administered from one week to four weeks. In one embodiment, azacitidine is administered from one week to three weeks. In one embodiment, azacitidine is administered from one week to two weeks. In one embodiment, azacitidine is administered once per day for about one week. In one embodiment, azacitidine is administered once per day for about two weeks. In one embodiment, azacitidine is administered once per day for about three weeks. In one embodiment, azacitidine is administered once per day for about four weeks. In one embodiment, azacitidine is administered once per day for about 6 weeks. In one embodiment, azacitidine is administered once per day for about 9 weeks. In one embodiment, azacitidine is administered once per day for about 12 weeks. In one embodiment, azacitidine is administered once per day for about 15 weeks. In one embodiment, azacitidine is administered once per day for about 18 weeks. In one embodiment, azacitidine is administered once per day for about 21 weeks. In one embodiment, azacitidine is administered once per day for about 26 weeks. In certain embodiments, azacitidine is administered intermittently. In certain embodiments, azacitidine is administered intermittently in the amount of between about 50 mg/m²/day and about 2,000 mg/m²/day. In certain embodiments, azacitidine is administered continuously. In certain embodiments, azacitidine is administered continuously in the amount of between about 50 mg/m²/day and about 1,000 mg/m²/day.

In certain embodiments, azacitidine is administered to a patient in cycles (e.g., daily administration for one week, then a rest period with no administration for up to three weeks). Cycling therapy involves the administration of an active agent for a period of time, followed by a rest for a period of time, and repeating this sequential administration. Cycling therapy can reduce the development of resistance, avoid or reduce the side effects, and/or improves the efficacy of the treatment.

In one embodiment, a method provided herein comprises administering azacitidine in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or greater than 40 cycles. In one embodiment, the median number of cycles administered in a group of patients is about 1. In one embodiment, the median number of cycles is about 2. In one embodiment, the median number of cycles is about 3. In one embodiment, the median number of cycles is about 4. In one embodiment, the median number of cycles is about 5. In one embodiment, the median number of cycles is about 6. In one embodiment, the median number of cycles is about 7. In one embodiment, the median number of cycles is about 8. In one embodiment, the median number of cycles is about 9. In one embodiment, the median number of cycles is about 10. In one embodiment, the median number of cycles is about 11. In one embodiment, the median number of cycles is about 12. In one embodiment, the median number of cycles is about 13. In one embodiment, the median number of cycles is about 14. In one embodiment, the median number of cycles is about 15. In one embodiment, the median number of cycles is about 16. In one embodiment, the median number of cycles is about 17. In one embodiment, the median number of cycles is about 18. In one embodiment, the median number of cycles is about 19. In one embodiment, the median number of cycles is about 20. In one embodiment, the median number of cycles is about 21. In one embodiment, the median number of cycles is about 22. In one embodiment, the median number of cycles is about 23. In one embodiment, the median number of cycles is about 24. In one embodiment, the median number of cycles is about 25. In one embodiment, the median number of cycles is about 26. In one embodiment, the median number of cycles is about 27. In one embodiment, the median number of cycles is about 28. In one embodiment, the median number of cycles is about 29. In one embodiment, the median number of cycles is about 30. In one embodiment, the median number of cycles is greater than about 30 cycles.

In one embodiment, azacitidine is administered to a patient at a dose provided herein over a cycle of 28 days which consists of a 7-day treatment period and a 21-day resting period. In one embodiment, azacitidine is administered to a patient at a dose provided herein each day from day 1 to day 7, followed with a resting period from day 8 to day 28 with no administration of azacitidine. In one embodiment, azacitidine is administered to a patient in cycles, each cycle consisting of a 7-day treatment period followed with a 21-day resting period. In particular embodiments, azacitidine is administered to a patient at a dose of about 50, about 60, about 70, about 75, about 80, about 90, or about 100 mg/m²/day, for 7 days, followed with a resting period of 21 days. In one embodiment, azacitidine is administered intravenously. In one embodiment, azacitidine is administered subcutaneously.

In other embodiments, azacitidine is administered orally in cycles. In one embodiment, azacitidine is administered daily in single or divided doses for about one week. In one embodiment, azacitidine is administered daily for about two weeks. In one embodiment, azacitidine is administered daily for about three weeks. In one embodiment, azacitidine is administered daily for about four weeks. In one embodiment, azacitidine is administered daily for about five weeks. In one embodiment, azacitidine is administered daily for about six weeks. In one embodiment, azacitidine is administered daily for about eight weeks. In one embodiment, azacitidine is administered daily for about ten weeks. In one embodiment, azacitidine is administered daily for about fifteen weeks. In one embodiment, azacitidine is administered daily for or about twenty weeks. The administration is followed by a rest period of about 1 day to about ten weeks. In one embodiment, the methods provided herein contemplate cycling treatments of about one week. In one embodiment, the methods provided herein contemplate cycling treatments of about two weeks. In one embodiment, the methods provided herein contemplate cycling treatments of about three weeks. In one embodiment, the methods provided herein contemplate cycling treatments of about four weeks. In one embodiment, the methods provided herein contemplate cycling treatments of about five weeks. In one embodiment, the methods provided herein contemplate cycling treatments of about six weeks. In one embodiment, the methods provided herein contemplate cycling treatments of about eight weeks. In one embodiment, the methods provided herein contemplate cycling treatments of about ten weeks. In one embodiment, the methods provided herein contemplate cycling treatments of about fifteen weeks. In one embodiment, the methods provided herein contemplate cycling treatments of about twenty weeks. In some embodiments, azacitidine is administered daily in single or divided doses for about one week. In one embodiment, azacitidine is administered daily for about two weeks. In one embodiment, azacitidine is administered daily for about three weeks. In one embodiment, azacitidine is administered daily for about four weeks. In one embodiment, azacitidine is administered daily for about five weeks. In one embodiment, azacitidine is administered daily for about six weeks. In one embodiment, the resting period of about 1, 3, 5, 7, 9, 12, 14, 16, 18, 20, 22, 24, 26, 28, 29, or 30 days. In some embodiments, the rest period is 1 day. In some embodiments, the rest period is 3 days. In some embodiments, the rest period is 7 days. In some embodiments, the rest period is 14 days. In some embodiments, the rest period is 28 days. The frequency, number and length of dosing cycles can be increased or decreased.

In one embodiment, azacitidine is administered subcutaneously for 7 days. In one embodiment, azacitidine is administered on days 1-7 of each 28-day cycle. In one embodiment, 75 mg/m²/day of azacitidine is administered on days 1-7 of each 28-day cycle.

In certain embodiments, the cytidine analog is 5-aza-2′-deoxycytidine (decitabine).

Decitabine is 4-amino-1-(2-deoxy-β-D-erythro-pentofuranosyl)-1,3,5-triazin-2(1H)one, also known as DACOGEN®. Its empirical formula is C₈H₁₂N₄O₄, the molecular weight is 228.21. Decitabine is a fine, white to almost white powder that is slightly soluble in ethanol/water (50/50), methanol/water (50/50) and methanol; sparingly soluble in water, and soluble in dimethylsulfoxide (DMSO).

DACOGEN® is approved for treatment in patients with myelodisplastic syndromes. It is supplied in a clear colorless glass vial as white sterile lyophilized powder for injection. Each 20 mL, as a single dose, glass vial contains 50 mg decitabine, 68 mg monobasic potassium phosphate (potassium dihydrogen phosphate) and 11.6 mg sodium hydrochloride.

In one embodiment, depending on the disease to be treated and the subject’s condition, decitabine may be administered by oral, parenteral (e.g., intramuscular, intraperitoneal, intravenous, CIV, intracistemal injection or infusion, subcutaneous injection, or implant), inhalation, nasal, vaginal, rectal, sublingual, or topical (e.g., transdermal or local) routes of administration. Decitabine may be formulated, alone or together with Compound 1 and/or one or more active agent(s), in suitable dosage unit with pharmaceutically acceptable excipients, carriers, adjuvants and vehicles, appropriate for each route of administration.

In one embodiment, decitabine is administered by, e.g., intravenous (IV), subcutaneous (SC) or oral routes. Certain embodiments herein provide co-administration of decitabine with Compound 1, or a pharmaceutically acceptable salt thereof, and/or one or more additional active agents to provide a therapeutic advantage in subjects in need thereof. Certain embodiments herein provide co-administration of decitabine with Compound 1, or a pharmaceutically acceptable salt thereof, and/or one or more additional active agents to provide a synergistic therapeutic effect in subjects in need thereof. The co-administered active agent(s) may be additional cancer therapeutic agents, as described herein (e.g., other DNA demethylating agents, BH3 mimetics, or standards of care).

In certain embodiments, treatment cycles comprise multiple doses of decitabine administered to a subject in need thereof over multiple days (e.g., 1, 2, 3, 4, 5, 6, 7, 8, 9, 10, 11, 12, 13, 14, or greater than 14 days). Suitable dosage amounts for the methods provided herein include, e.g., therapeutically effective amounts and prophylactically effective amounts. For example, in certain embodiments, the amount of decitabine administered in the methods provided herein may range, e.g., between about 5 mg/m²/day and about 500 mg/m²/day. In certain embodiments, the amount of decitabine is between about 5 mg/m²/day and about 300 mg/m²/day. In certain embodiments, the amount of decitabine is between about 5 mg/m²/day and about 100 mg/m²/day. In certain embodiments, the amount of decitabine is between about 5 mg/m²/day and about 50 mg/m²/day. In certain embodiments, the amount of decitabine is between about 10 mg/m²/day and about 50 mg/m²/day. In certain embodiments, the amount of decitabine is between about 10 mg/m²/day and about 25 mg/m²/day. In certain embodiments, the particular dosage is about 5 mg/m²/day. In one embodiment, the particular dosage is about 10 mg/m²/day. In one embodiment, the particular dosage is about 15 mg/m²/day. In one embodiment, the particular dosage is about 20 mg/m²/day. In one embodiment, the particular dosage is about 25 mg/m²/day. In one embodiment, the particular dosage is about 30 mg/m²/day. In one embodiment, the particular dosage is about 35 mg/m²/day. In one embodiment, the particular dosage is about 40 mg/m²/day. In one embodiment, the particular dosage is about 45 mg/m²/day. In one embodiment, the particular dosage is about 50 mg/m²/day. In one embodiment, the particular dosage is about 55 mg/m²/day. In one embodiment, the particular dosage is about 60 mg/m²/day. In one embodiment, the particular dosage is about 70 mg/m²/day. In one embodiment, the particular dosage is about 80 mg/m²/day. In one embodiment, the particular dosage is about 90 mg/m²/day. In one embodiment, the particular dosage is about 100 mg/ m²/day.

In one embodiment, the amount of decitabine in the pharmaceutical composition or dosage form provided herein may range, e.g., between about 5 mg/m²/day and about 500 mg/m²/day. In certain embodiments, the amount of decitabine is between about 5 mg/m²/day and about 300 mg/m²/day. In certain embodiments, the amount of decitabine is between about 5 mg/m²/day and about 100 mg/m²/day. In certain embodiments, the amount of decitabine is between about 5 mg/m²/day and about 50 mg/m²/day. In certain embodiments, the amount of decitabine is between about 10 mg/m²/day and about 50 mg/m²/day. In certain embodiments, the amount of decitabine is between about 10 mg/m²/day and about 25 mg/m²/day. In certain embodiments, the particular dosage is about 5 mg/m²/day. In one embodiment, the particular dosage is about 10 mg/m²/day. In one embodiment, the particular dosage is about 15 mg/m²/day. In one embodiment, the particular dosage is about 20 mg/m²/day. In one embodiment, the particular dosage is about 25 mg/m²/day. In one embodiment, the particular dosage is about 30 mg/m²/day. In one embodiment, the particular dosage is about 35 mg/m²/day. In one embodiment, the particular dosage is about 40 mg/m²/day. In one embodiment, the particular dosage is about 45 mg/m²/day. In one embodiment, the particular dosage is about 50 mg/m²/day. In one embodiment, the particular dosage is about 55 mg/m²/day. In one embodiment, the particular dosage is about 60 mg/m²/day. In one embodiment, the particular dosage is about 70 mg/m²/day. In one embodiment, the particular dosage is about 80 mg/m²/day. In one embodiment, the particular dosage is about 90 mg/m²/day. In one embodiment, the particular dosage is about 100 mg/ m²/day. In one embodiment, decitabine can be delivered as a single dose such as, e.g., a single bolus injection, or oral tablets or pills; or over time such as, e.g., continuous infusion over time or divided bolus doses over time. In one embodiment, decitabine can be administered repetitively if necessary, for example, until the patient experiences stable disease or regression, or until the patient experiences disease progression or unacceptable toxicity. Stable disease or lack thereof is determined by methods known in the art such as evaluation of patient’s symptoms, physical examination, visualization of the tumor that has been imaged using X-ray, CAT, PET, or MRI scan and other commonly accepted evaluation modalities.

In one embodiment, decitabine can be administered once daily or divided into multiple daily doses such as twice daily, three times daily, and four times daily. In one embodiment, the administration can be continuous (i.e., daily for consecutive days or every day), intermittent, e.g., in cycles (i.e., including days, weeks, or months of rest when no drug is administered). In one embodiment, decitabine is administered daily, for example, once or more than once each day for a period of time. In one embodiment, decitabine is administered every 8 hours. In one embodiment, decitabine is administered daily for an uninterrupted period of at least 3 days. In some embodiments, decitabine is administered up to 5 days. In one embodiment, decitabine is administered intermittently, i.e., stopping and starting at either regular or irregular intervals. In one embodiment, decitabine is administered on alternate days. In one embodiment, decitabine is administered in cycles (e.g., administered daily or continuously for a certain period interrupted with a rest period). In one embodiment, decitabine is administered daily for a period of at least 3 days (e.g., for 3 days or for 5 days), then a rest period with no administration for up to 6 weeks; or e.g., daily administration for at least three days, then a rest period with no administration for up to 6 weeks). Cycling therapy can reduce the development of resistance, avoid or reduce the side effects, and/or improves the efficacy of the treatment.

In one embodiment, a method provided herein comprises administering decitabine in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or greater than 40 cycles. In one embodiment, the median number of cycles administered in a group of patients is about 1. In one embodiment, the median number of cycles is about 2. In one embodiment, the median number of cycles is about 3. In one embodiment, the median number of cycles is about 4. In one embodiment, the median number of cycles is about 5. In one embodiment, the median number of cycles is about 6. In one embodiment, the median number of cycles is about 7. In one embodiment, the median number of cycles is about 8. In one embodiment, the median number of cycles is about 9. In one embodiment, the median number of cycles is about 10. In one embodiment, the median number of cycles is about 11. In one embodiment, the median number of cycles is about 12. In one embodiment, the median number of cycles is about 13. In one embodiment, the median number of cycles is about 14. In one embodiment, the median number of cycles is about 15. In one embodiment, the median number of cycles is about 16. In one embodiment, the median number of cycles is about 17. In one embodiment, the median number of cycles is about 18. In one embodiment, the median number of cycles is about 19. In one embodiment, the median number of cycles is about 20. In one embodiment, the median number of cycles is about 21. In one embodiment, the median number of cycles is about 22. In one embodiment, the median number of cycles is about 23. In one embodiment, the median number of cycles is about 24. In one embodiment, the median number of cycles is about 25. In one embodiment, the median number of cycles is about 26. In one embodiment, the median number of cycles is about 27. In one embodiment, the median number of cycles is about 28. In one embodiment, the median number of cycles is about 29. In one embodiment, the median number of cycles is about 30. In one embodiment, the median number of cycles is greater than about 30 cycles.

In one embodiment, decitabine is administered at a dose of 15 mg/m² by continuous intravenous infusion over 3 hours repeated every 8 hours for 3 days. In one embodiment, this cycle is repeated every 6 weeks.

In one embodiment, decitabine is administered at a dose of 20 mg/m² by continuous intravenous infusion over 1 hour repeated daily for 5 days. In one embodiment, this cycle is repeated every 4 weeks.

In one embodiment, the first anti-cancer agent and the at least one second anti-cancer agent can be administered simultaneously in the same or different formulations. In an alternative embodiment, the first anti-cancer agent and the at least one second anti-cancer agent can be administered sequentially, i.e., at different points in time.

In some embodiments, the first anti-cancer agent is a pharmaceutically acceptable salt of the compound of Formula (I). In one embodiment, the first anti-cancer agent is the tris(hydroxymethyl)aminomethane (“Tris”) salt of the compound of Formula (I). In another embodiment, the first anti-cancer agent is the sodium salt of the compound of Formula (I).

In some embodiments, the compound of Formula (I) or a pharmaceutically acceptable salt thereof is a solid state or crystalline form of the compound of Formula (I), as described in PCT/US2019/067897, filed Dec. 20, 2019. The entire teachings of PCT/US2019/067897 are incorporated herein by reference. In one embodiment, the compound of Formula (I) is Form A, B, C, or D of the crystalline form of the compound of Formula (I). In one embodiment, the compound of Formula (I) is Form A, B, or C of the crystalline form of the Tris salt of the compound of Formula (I). In one embodiment, the compound of Formula (I) or the Tris salt thereof is in amorphous form.

In one embodiment, the dosing regimen for the cancer treatment method described in the foregoing paragraphs comprises administering an effective amount of the compound of Formula (I) or a pharmaceutically acceptable salt thereof (e.g., the Tris salt of the compound of Formula (I) and the sodium salt of the compound of Formula (I)) once a week in one or more 28-day or 4-week cycles.

In one embodiment, the dosing regimen for the cancer treatment method described in the foregoing paragraphs comprises administering an effective amount of the compound of Formula (I) or a pharmaceutically acceptable salt thereof (e.g., the Tris salt of the compound of Formula (I) and the sodium salt of the compound of Formula (I)) for a period of 2 to 5 days each week, with 1 cycle of therapy defined as 4 consecutive weeks of treatment

In one embodiment, the dosing regimen for the cancer treatment method described in the foregoing paragraphs comprises administering an effective amount of the compound of Formula (I) or a pharmaceutically acceptable salt thereof (e.g., the Tris salt of the compound of Formula (I) and the sodium salt of the compound of Formula (I)) for a period of 2 to 5 consecutive days followed by a 2 to 5 day break each week, in one or more 28-day or 4-week cycle.

In one embodiment, the dosing regimen for the cancer treatment method described in the foregoing paragraphs comprises administering an effective amount of the compound of Formula (I) or a pharmaceutically acceptable salt thereof (e.g., the Tris salt of the compound of Formula (I) and the sodium salt of the compound of Formula (I)) for 2 consecutive days followed by a 5-day break each week, in one or more 28-day or 4-week cycles.

In one embodiment, the dosing regimen for the cancer treatment method described in the foregoing paragraphs comprises administering an effective amount of the compound of Formula (I) or a pharmaceutically acceptable salt thereof (e.g., the Tris salt of the compound of Formula (I) and the sodium salt of the compound of Formula (I)) for 3 consecutive days followed by a 4-day break each week, in one or more 28-day or 4-week cycles.

In one embodiment, the dosing regimen for the cancer treatment method described in the foregoing paragraphs comprises administering an effective amount of the compound of Formula (I), or a pharmaceutically acceptable salt thereof (e.g., the Tris salt of the compound of Formula (I) and the sodium salt of the compound of Formula (I)) for 4 consecutive days followed by a 3-day break each week, in one or more 28-day or 4-week cycles.

In one embodiment, the dosing regimen for the cancer treatment method described in the foregoing paragraphs comprises administering an effective amount of the compound of Formula (I), or a pharmaceutically acceptable salt thereof (e.g., the Tris salt of the compound of Formula (I) and the sodium salt of the compound of Formula (I)) for 5 consecutive days followed by a 2-day break each week, in one or more 28-day or 4-week cycles.

In one embodiment, the dosing regimen for the cancer treatment method described in the foregoing paragraphs comprises administering an effective amount of the compound of Formula (I), or a pharmaceutically acceptable salt thereof (e.g., the Tris salt of the compound of Formula (I) and the sodium salt of the compound of Formula (I)) for a period of 2 to 5 consecutive days followed by a 2 to 5 day break each week, in one or more 28-day or 4-week cycle, where the number of consecutive days of treatment is increased in at least one of the weeks of the 28-day or 4-week cycle, in one or more 28-day or 4-week cycles.

In another embodiment, the dosing regimen for the cancer treatment method described in the foregoing paragraphs comprises administering an effective amount of the compound of Formula (I), or a pharmaceutically acceptable salt thereof (e.g., the Tris salt of the compound of Formula (I) and the sodium salt of the compound of Formula (I)) for a period of 2 to 5 consecutive days followed by a 2 to 5 day break each week, in one or more 28-day or 4-week cycle, where the number of consecutive days of treatment is decreased in at least one of the weeks of the 28-day or 4-week cycle, in one or more 28-day or 4-week cycles.

In another embodiment, the effective amount of the compound of Formula (I), or a pharmaceutically acceptable salt thereof (e.g., the Tris salt of the compound of Formula (I) and the sodium salt of the compound of Formula (I)) is administered to the subject for 2 consecutive days followed by a 5-day break for the first week of a 28-day or 4-week cycle, where the number of consecutive days of treatment is increased in at least one of the weeks of the 28-day or 4-week cycle.

In another embodiment, the effective amount of the compound of Formula (I), or a pharmaceutically acceptable salt thereof (e.g., the Tris salt of the compound of Formula (I) and the sodium salt of the compound of Formula (I)) is administered to the subject for 2 consecutive days followed by a 5-day break for the first week of a 28-day or 4-week cycle, where the number of consecutive days of treatment is decreased in at least one of the weeks of a 28-day or 4-week cycle.

In another embodiment, the effective amount of the compound of Formula (I), or a pharmaceutically acceptable salt (e.g., the Tris salt of the compound of Formula (I) and the sodium salt of the compound of Formula (I)) is administered to the subject for 3 consecutive days followed by a 4-day break for the first week of a 28-day or 4-week cycle, where the number of consecutive days of treatment is increased in at least one of the weeks of a 28-day or 4-week cycle.

In another embodiment, the effective amount of the compound of Formula (I), or a pharmaceutically acceptable salt thereof (e.g., the Tris salt of the compound of Formula (I) and the sodium salt of the compound of Formula (I)) is administered to the subject for 3 consecutive days followed by a 4-day break for the first week of a 28-day or 4-week cycle, where the number of consecutive days of treatment is decreased in at least one of the weeks of a 28-day or 4-week cycle.

In another embodiment, the effective amount of the compound of Formula (I), or a pharmaceutically acceptable salt thereof (e.g., the Tris salt of the compound of Formula (I) and the sodium salt of the compound of Formula (I)) is administered to the subject for 4 consecutive days followed by a 3-day break for the first week of a 28-day or 4-week cycle, where the number of consecutive days of treatment is increased in at least one of the weeks of the 28-day or 4-week cycle.

In another embodiment, the effective amount of the compound of Formula (I), or a pharmaceutically acceptable salt thereof (e.g., the Tris salt of the compound of Formula (I) and the sodium salt of the compound of Formula (I)) is administered to the subject for 4 consecutive days followed by a 3-day break for the first week of a 28-day or 4-week cycle, where the number of consecutive days of treatment is decreased in at least one of the weeks of a 28-day or 4-week cycle.

In another embodiment, the effective amount of the compound of Formula (I), or a pharmaceutically acceptable salt thereof (e.g., the Tris salt of the compound of Formula (I) and the sodium salt of the compound of Formula (I)) is administered to the subject for 5 consecutive days followed by a 2-day break for the first week of a 28-day or 4-week cycle, where the number of consecutive days of treatment is increased in at least one of the weeks of the 28-day or 4-week cycle.

In another embodiment, the effective amount of the compound of Formula (I), or a pharmaceutically acceptable salt thereof (e.g., the Tris salt of the compound of Formula (I) and the sodium salt of the compound of Formula (I)) is administered to the subject for 5 consecutive days followed by a 2-day break for the first week of a 28-day or 4-week cycle, where the number of consecutive days of treatment is decreased in at least one of the weeks of a 28-day or 4-week cycle.

In one embodiment, the dosing regimen for the cancer treatment method described in the foregoing paragraphs comprises administering an effective amount of the compound of Formula (I), or a pharmaceutically acceptable salt thereof (e.g., the Tris salt of the compound of Formula (I) and the sodium salt of the compound of Formula (I)) orally. In one embodiment, the effective amount of the compound of Formula (I) or a pharmaceutically acceptable salt thereof (e.g., the Tris salt of the compound of Formula (I) and the sodium salt of the compound of Formula (I)) is administered as an oral capsule. In another embodiment, the effective amount of the compound of Formula (I), or a pharmaceutically acceptable salt thereof (e.g., the Tris salt of the compound of Formula (I) and the sodium salt of the compound of Formula (I)) is administered as an oral tablet.

In one embodiment, the dosing regimen for the cancer treatment method described in the foregoing paragraphs comprises administering an effective amount of the compound of Formula (I), or a pharmaceutically acceptable salt thereof (e.g., the Tris salt of the compound of Formula (I) and the sodium salt of the compound of Formula (I)) once daily (QD). In another embodiment, the dosing regimen for the cancer treatment method described in the foregoing paragraphs comprises administering an effective amount of the compound of Formula (I), or a pharmaceutically acceptable salt thereof (e.g., the Tris salt of the compound of Formula (I) and the sodium salt of the compound of Formula (I)) twice daily (BID).

The same dosing regimens described above also apply to the second anti-cancer agent if it is administered concurrently with the first anti-cancer agent (i.e., the compound of Formula (I), or a pharmaceutically acceptable salt thereof) either in the same pharmaceutical formulation or in separate pharmaceutical formulations. If the second anti-cancer agent is not administered concurrently with the first anti-cancer agent, those skilled in the art will be able to determine suitable dosing regimens using routine experimentation.

### Compositions

In a further aspect, the subject application is directed to a pharmaceutical composition comprising a first anti-cancer agent and at least one second anti-cancer agent, wherein the pharmaceutical composition additionally comprises an excipient, wherein the first anti-cancer agent is represented by Formula (I):

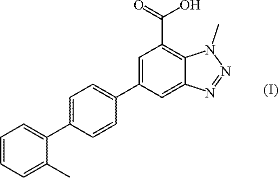 [SMILES: Cc1ccccc1-c1ccc(-c2cc(C(=O)O)c3c(c2)nnn3C)cc1]

or a pharmaceutically acceptable salt thereof; and wherein the at least one second anti-cancer agent is selected from the group consisting of a BH3 mimetic and a DNA demethylating agent, or a combination thereof. In another embodiment, the at least one second anti-cancer agent is a combination of a BH3 mimetic and a DNA demethylating agent. In another embodiment, the at least one second anti-cancer agent is a BH3 mimetic. In one embodiment, the BH3 mimetic is selected from the group consisting of venetoclax, BGB-11417, navitoclax (ABT-263), LP-108, S55746, S65487, AT-101, APG-1252, obatoclax, APG2575, and BCL-201. In another embodiment, the BH3 mimetic is ventoclax. In one embodiment, the at least one second anti-cancer agent is a DNA demethylating agent. In one embodiment, the DNA demethylating agent is a cytidine analogue. In another embodiment, the the DNA demethylating agent is selected from the group consisting of 5-azacitidine (azacitidine), 5-aza-2′-deoxycytidine (decitabine), 1-β-D-arabinofuranosylcytosine (cytarabine or ara-C), pseudoiso-cytidine (psi ICR), 5-fluoro-2′-deoxycytidine (FCdR), 2′-deoxy-2′,2′-difluorocytidine (gemcitabine), 5-aza-2′-deoxy-2′,2′-difluorocytidine; 5-aza-2′-deoxy-2′-fluorocytidine; 1-β-D-ribofuranosyl-2(1H)-pyrimidinone (Zebularine), 2′,3′-dideoxy-5-fluoro-3′-thiacytidine (emtriva), 2′-cyclocytidine (Ancitabine), 1-β-D-arabinofuranosyl-5-azacytosine (fazarabine or ara-AC), 6-azacitidine (6-aza-CR), 5,6-dihydro-5-azacitidine (dH-aza-CR), N4 pentyloxy-carbonyl-5′-deoxy-5-fluorocytidine (capecitabine), N4 octadecyl-cytarabine, and elaidic acid cytarabine. In another embodiment, the DNA demethylating agent is selected from the group consisting of azacitidine and cytarabine. In another embodiment, the DNA demethylating agent is selected from the group consisting of azacitidine. In another embodiment, the DNA demethylating agent is selected from the group consisting of decitabine.

In some embodiments, the first anti-cancer agent is a pharmaceutically acceptable salt of the compound of Formula (I). In one embodiment, the first anti-cancer agent is the tris(hydroxymethyl)aminomethane salt of the compound of Formula (I). In another embodiment, the first anti-cancer agent is the sodium salt of the compound of Formula (I).

The first anti-cancer agent and/or second anti-cancer agent may be formulated together with a pharmaceutically acceptable carrier, adjuvant, or vehicle into pharmaceutical compositions prior to being administered to a subject.

The term “pharmaceutically acceptable carrier, adjuvant, or vehicle” refers to a carrier, adjuvant, or vehicle that may be administered to a subject, together with the compound of Formula (I), or a pharmaceutically acceptable salt thereof, and/or a second anti-cancer agent and which does not destroy the pharmacological activity thereof and is nontoxic when administered in doses sufficient to deliver a therapeutic amount of the compound. Those skilled in the art would know how to prepare formulations suitable for administration. The second anti-cancer agent optionally is part of the same formulation for the first anti-cancer agent (i.e., the compound of Formula (I), or a pharmaceutically acceptable salt thereof), if it is administered concurrently with the first anti-cancer agent, or, alternatively, can be administered separately.

Pharmaceutically acceptable carriers, adjuvants and vehicles that may be used in the pharmaceutical compositions include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of the compound of Formula (I), or a pharmaceutically acceptable salt thereof, and/or a second anti-cancer agent.

The pharmaceutical compositions may be administered orally, parenterally, by inhalation spray, topically, rectally, nasally, buccally, vaginally or via an implanted reservoir, preferably by oral administration or administration by injection. The pharmaceutical compositions may contain any conventional non-toxic pharmaceutically acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer’s solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions may also be administered in the form of suppositories for rectal administration. These compositions can be prepared by mixing the compound of Formula (I), or a pharmaceutically acceptable salt thereof, and/or a second anti-cancer agent with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions may be administered topically to the skin. The pharmaceutical composition should be formulated with a suitable ointment containing the active components suspended or dissolved in a carrier. Carriers for topical administration of the compounds of one aspect of this application include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of one aspect of this application may also be topically applied to the lower intestinal tract by rectal suppository formulation or in a suitable enema formulation. Topical transdermal patches are also included in one aspect of this application.

The pharmaceutical compositions may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The amount of active ingredient that may be combined with one or more pharmaceutical excipients to produce a single dosage form will vary depending upon the patient treated and the particular mode of administration. A typical preparation will contain from about 5% to about 95% active compound (w/w). Alternatively, such preparations contain from about 20% to about 80% active compound. In certain embodiments, the pharmaceutical composition comprises between about 10 mg to about 1500 mg of the compound of Formula (I) or a pharmaceutically acceptable salt thereof (based on the weight of the free form of the compound of Formula (I), apart from the weight of any conformer, salt former, water of hydration, solvent of solvation and the like). In some embodiments, the pharmaceutical composition comprises between about 90 mg and about 240 mg; between about 100 mg and about 240 mg; between about 10 mg and about 500 mg; or between about 10 mg and about 1000 mg of the compound of Formula (I) or a pharmaceutically acceptable salt. In some embodiments, the pharmaceutical composition comprises about 10 mg, about 25 mg, about 50 mg, about 75 mg, about 90 mg, about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 275 mg, about 280 mg, about 290 mg, or about 300 mg of the compound of Formula (I) or a pharmaceutically acceptable salt thereof. In some embodiments, the pharmaceutical composition comprises about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, or about 1450 mg, or about 1500 mg. In certain embodiments, the pharmaceutical composition is in the form of an orally acceptable dosage form, such as for example a capsule, and comprises about 50 mg, about 75 mg, about 90 mg, about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 275 mg, about 280 mg, about 290 mg, or about 300 mg of the Tris salt of the compound of Formula (I).

As used herein, the terms “about” and “approximately” when used in combination with a numeric value or range of values mean the value or range of values may deviate to an extent deemed reasonable to one of ordinary skill in the art.

The compound of Formula (I) and formulations thereof can be prepared according to methods described in WO 2014/128669, WO 2019/164794, PCT/US2019/067897, and U.S. Pat. No. 9,630,932, the contents of which are incorporated herein by reference.

### EXAMPLES

### Example 1

### Cell Viability Following Incubation With Compound 1 Alone and in Combination With Cytarabine, Azacitidine, or Venetoclax

Cell growth assays (non-screen format) were performed using CTG as a readout of viable cells. For CTG assays, cells were plated in 96-well tissue culture plates (Corning) at 10,000 cells/well in media containing 5 µM uridine. Cells were incubated with Compound 1 or DMSO control in combination with cytarabine, azacitidine, or venetoclax for 96 hours in AML and DLBCL cell lines (MOLM-13, THP-1, NOMO-1, SUDHL-2, SUDHL-4, and Pfeiffer). CTG readings were performed at T0 and at T96 using a Molecular Devices SpectraMAX Paradigm plate reader. Relative growth rates (µ/µmax) were calculated using the CTG time 0 (T0) and 96-hour (T96) ATP measurements according to the following formula:

$$\frac{μ}{{μ}_{m a x}} = \frac{\frac{l n \frac{T}{{V}_{0}}}{T i m e}}{\frac{l n \frac{V}{{V}_{0}}}{T i m e}}$$

where T is the signal measure for the drug-treated arm at T96 and V is the DMSO vehicle treated control measure at T96. V0 is the vehicle-treated control measure at T0. A value of 1 indicates no growth inhibition, 0 indicates complete growth inhibition, and a value <0 indicates cell death.

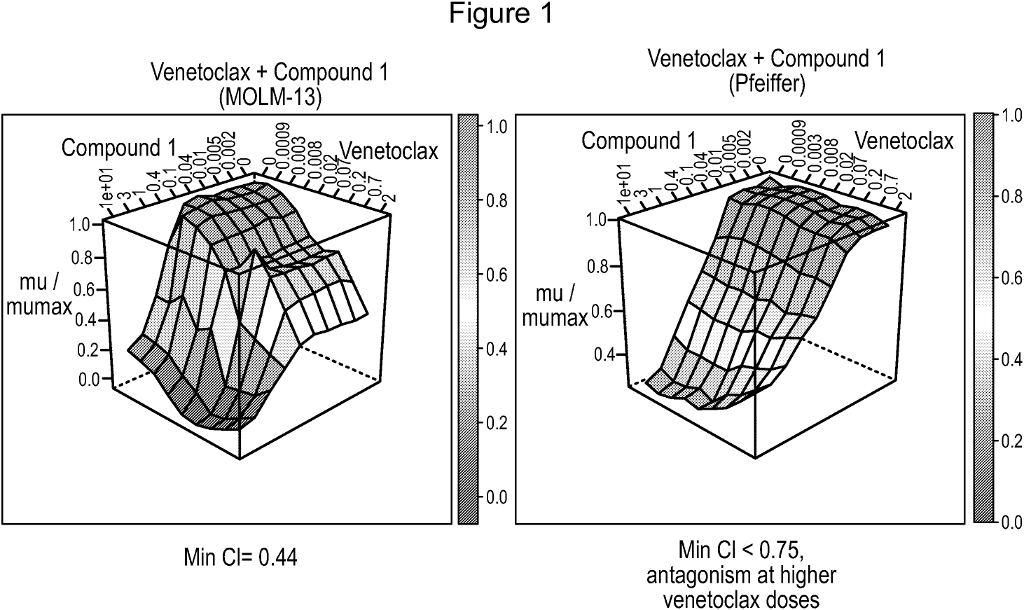

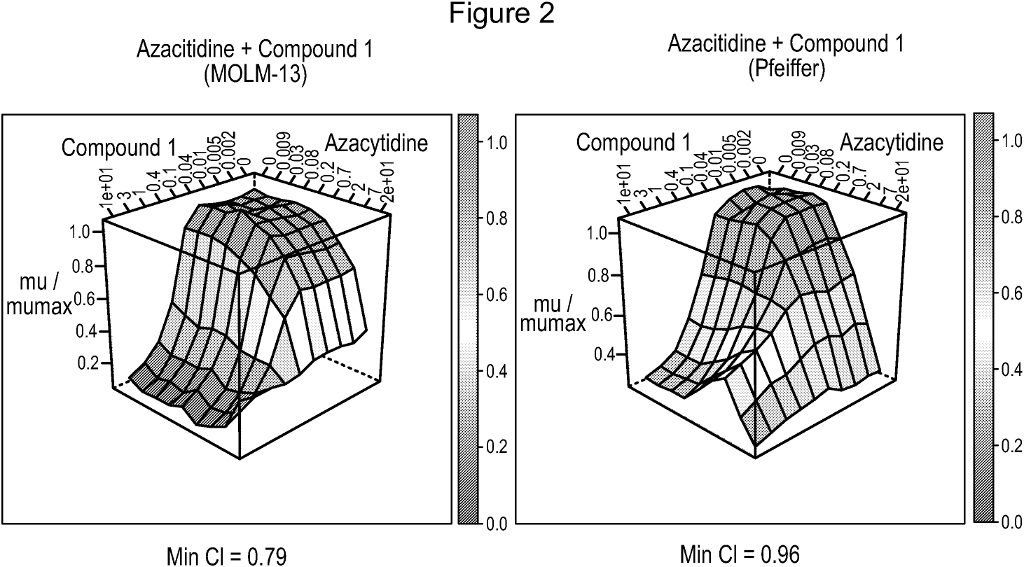

As shown in FIG. 1, treatment of acute myeloid leukemia (AML) and diffuse large B-cell lymphoma (DLBCL) cell lines with Compound 1 and the BH3-mimetic venetoclax resulted in synergistic growth inhibition at certain drug concentrations in MOLM-13 and Pfeiffer cell lines (CI=0.44 in MOLM-13 and CI<0.75 in Pfeiffer). In those same cell lines as shown in FIG. 2, the DNA demethylating agent azacitidine resulted in modest synergy in growth inhibition when combined with Compound 1 in the AML cell line (MOLM-13; CI=0.79), but not the DLBCL cell line (Pfeiffer; CI=0.96). The combination of Compound 1 and venetoclax or azacitidine did not demonstrate obvious synergistic growth inhibition in the THP-1, NOMO-1, SUDHL2, and SUDHL4 cell lines (data not shown), though those cell lines did not have sufficient single agent sensitivity to Compound 1 in the growth media used for the experiments in order to perform the synergy analysis.

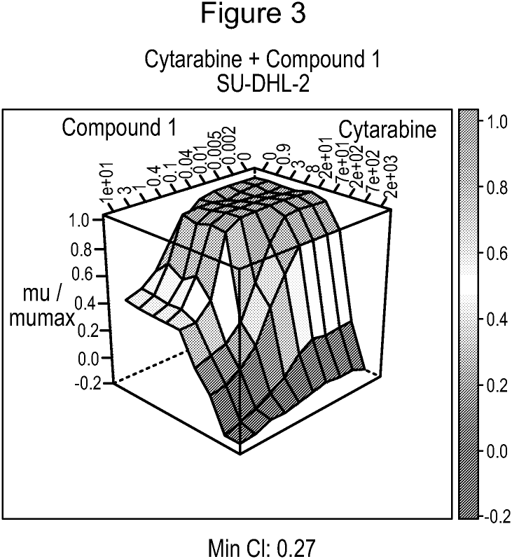

A leukemia standard of care, cytarabine, was also tested in combination with Compound 1. The combination of Compound 1 and cytarabine resulted in synergistic growth inhibition at certain drug concentrations in the SUDHL2 cell line (FIG. 3), but not in any of the other tested cell lines (data not shown).

### Example 2

### Compound 1 With Azacitidine Results in Synergistic Growth Inhibition in AML and DLBCL Cell Lines

Dose response matrix screening (9×9 format) in 384-well plates was carried out with Compound 1 in combination independently with azacitidine. Growth assays were carried out in vendor-recommended media with CTG readings performed at T0 and T96. To measure combination effects in excess of additivity, a scalar measure derived by Horizon Discovery to characterize the strength of synergistic interaction (Synergy Score) was utilized as described (Schaffer, et al). Synergy scores >2.4 are considered to be substantially above the background noise of the assay. Scores from 2.4-9.3 are considered mild to moderate synergy, while scores >9.3 are considered strong synergy. Potency shifting was also scored using combination index (CI) which is calculated as

$$C I = \frac{{C}_{X}}{E {C}_{X}} + \frac{{C}_{Y}}{E {C}_{Y}}$$

where

$$\frac{{C}_{X}}{E {C}_{X}}$$

for a particular data point is the ratio of the X compound’s measured concentration to its effective concentration at the chosen effect level. The CI is a rough estimate of how much drug was needed in combination relative to the single agent doses required to achieve the chosen effect level. CI values 0.5-0.7 are typical for in vitro measurements of current clinical combinations. The CI error (σCI) is calculated using standard error propagation through the CI calculation based on the isobologram errors. Best CI is the CI with the largest signal-to-noise ratio level (1-CI)/ σCI.

Combination of Compound 1 with azacitidine resulted in synergistic growth inhibition in two out of three AML cell lines and two out of four DLBCL cell lines. See Table 1.

TABLE 1

| | | Azacitidine | |
|---|---|---|---|
| | | Synergy Score | Best CI |
| AML | HL-60 | 0.9 | 0.5 |
| | MOLM13 | 7.0 | 0.3 |
| | MV-4-11 | 7.1 | 0.4 |
| DLBCL | DB | 1.6 | 0.7 |
| | DOHH-2 | 7.7 | 0.6 |
| | Pfeiffer | 2.4 | 1.1 |
| | SU-DHL-5 | 3.4 | 0.8 |

### Example 3

### Tolerability and Growth Inhibition Study of Compound 1 With Venetoclax In Tumor-Bearing Mice

### Venetoclax Single Agent Dose Response Study

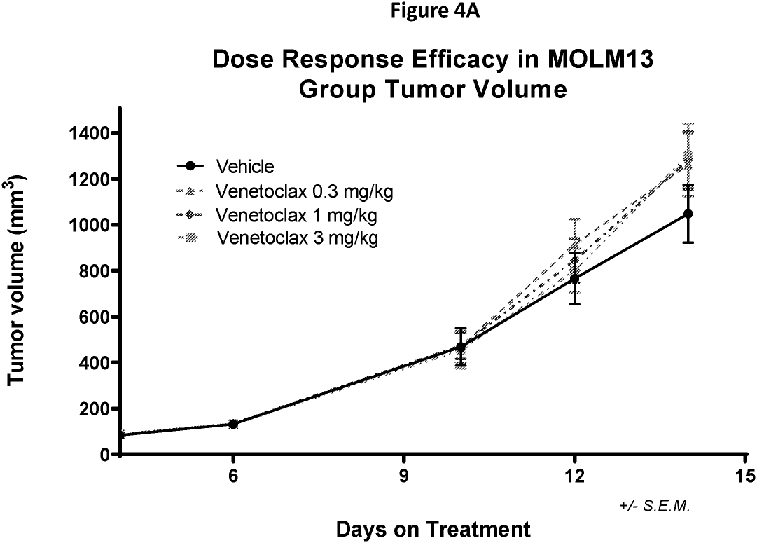

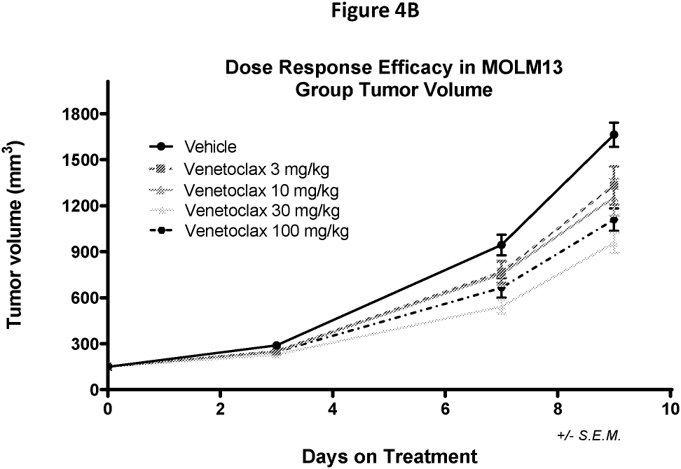

Efficacy of venetoclax in an AML xenograft model (MOLM-13) was assessed as part of in vivo experiments with Compound 1 and venetoclax. Tumor-bearing mice (n = 15/group) were treated with vehicle or venetoclax at varying doses with venetoclax administered twice daily (QD) PO. FIGS. 4A and 4B show the dose response efficacy curves of venetoclax at 0.3, 1, 3, 10, 30, and 100 mg/kg.

### Tolerability Study (Compound 1 & Venetoclax)

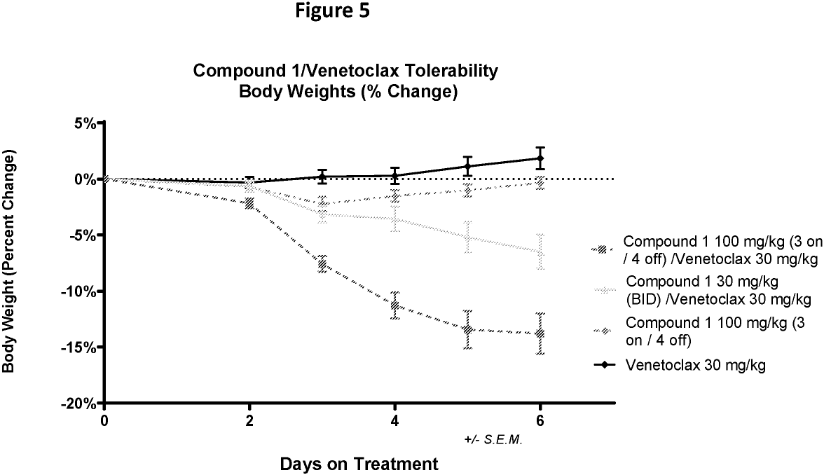

The feasibility of the combination of Compound 1 and venetoclax was assessed in tolerability studies conducted at various doses of Compound 1 in combination with venetoclax. Tolerability was determined by assessing overall body conditions as well as body weight assessments from each animal and each group as a whole. Combinations were considered tolerated when at least one animal in a given group had greater than a 20% body weight loss when compared to its weight at the start of treatment, or when a group’s average had greater than 15% body weight loss. In one study, Compound 1 was not tolerated when given BID at 100 mg/kg in combination with 30 mg/kg of venetoclax in CB17 SCID mice. In another study in CB17 SCID mice, 30 mg/kg of Compound 1 given BID in combination with 30 mg/k of Venetoclax was tolerated as shown by the average body weight percent change curves in FIG. 5.

### Growth Inhibition Study (AML) (Compound 1 & Venetoclax)

The combination of Compound 1 with venetoclax was shown to be beneficial in the MOLM13 xenograft model of AML. 5 × 10⁶ MOLM13 cells were implanted subcutaneously into 5-6 week-old female CB17 SCID mice in serum free media with Matrigel 1:1. Tumor volume and body weights were monitored twice a week until the tumors reached ~ 150 mm³. Once the tumor burden was reached, mice were randomized into six groups—(1) vehicle (n = 15 mice), (2) Compound 1 (n = 15 mice), (3) venetoclax (n = 15 mice), (4) first combination of Compound 1 and Venetoclax (n = 15 mice, (5) second combination of Compound 1 and venetoclax (n = 15 mice), and (6) a satellite arm (n = 10 mice)-and dosed as outlined in Table 7 below. In the satellite arm, tumor volume and body weights were monitored twice a week until the tumors reached ~500 mm³. Once this tumor burden was reached, mice were randomized into two groups—vehicle and Compound 1. Groups in the satellite arm were dosed for 2 doses via oral gavage at 10 ml/kg. For groups 4 and 5 wherein Compound 1 and venetoclax were co-administered on the same day, Compound 1 was dosed at T=0 hr and T=12 hr and venetoclax dosed at T=6 hr. At the end of study, tumor, plasma, and liver tissues were collected at the indicated timepoints post-last dose for PK and biomarker analyses. Compound 1 was formulated with 0.5% Methyl Cellulose/deionized water. Venetoclax was formulated with 60% Phosal 50, 30% PEG 400, and 10% ethanol and stored at 4° C.

TABLE 7

| Group | Cpd 1 Dose | Cpd 1 Schedule | Venetoclax Dose | Venetoclax Schedule | End of Study: Time Points for PK |
|---|---|---|---|---|---|
| 1 | VEHICLE - 60% Phosal 50 + 30% PEG 400+ 10% Ethanol | | | | End of Study- Pre, 1, 4, 7 & 24 hr (N=3/TP) |
| 2 | 30 mg/kg | BID | N/A | N/A | |
| 3 | N/A | N/A | 30 mg/kg | QD | |
| 4 | 30 mg/kg | BID | 30 mg/kg | QD | |
| 5 | 100 mg/kg | 2 days on, 1 day off (BID) | 30 mg/kg | QD | |
| 6* | 100 mg/kg | BID | N/A | N/A | End of Study- 4 hr |

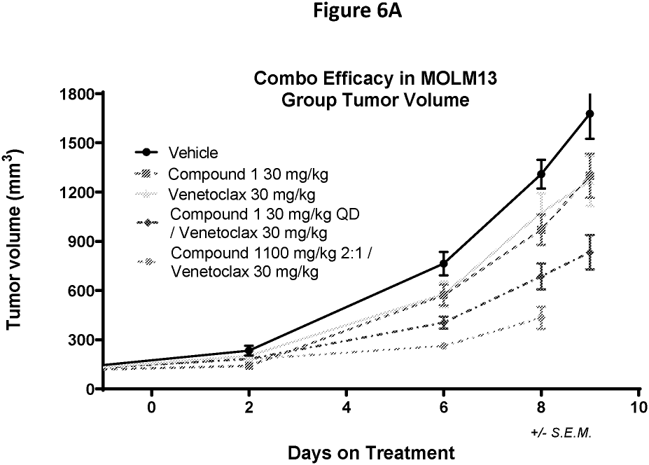

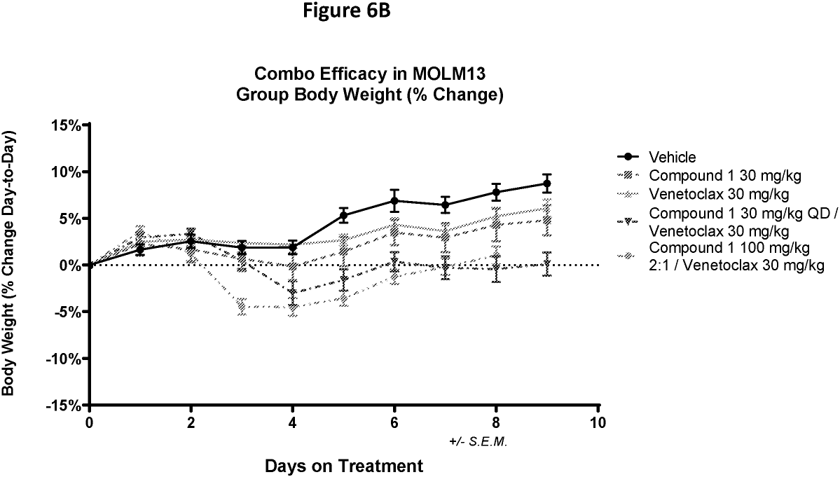

The combination of Compound 1 administered at 30 mg/kg (QD) and venetoclax showed a two-fold increase in tumor growth inhibition benefit over each single agent. (FIG. 6A; Table 8.) When Compound 1 was used at 100 mg/kg but given on a holiday schedule (2 days on, 1 day off), tumor growth inhibition increased approximately three-fold over each single agent. (FIG. 6A; Table 8.) The combinations of Compound 1 and venetoclax in the study were all well tolerated. (FIG. 6B).

TABLE 8

| Treatment | Tumor Growth Inhibition |
|---|---|
| Compound 1 (30 mg/kg) | 24% |
| Venetoclax (30 mg/kg) | 27% |
| Compound 1 (30 mg/kg QD) + Venetoclax | 54% |
| Compound 1 (100 mg/kg holiday schedule) + Venetoclax | 74% |

### Growth Inhibition Study (DLBCL) (Compound 1 & Venetoclax)

The combination of Compound 1 with venetoclax was shown to be beneficial in the OCILY19 xenograft model of DLBCL. 5 × 10⁶ OCILY cells were implanted subcutaneously into 5-6 week-old female CB17 SCID mice in serum free media with Matrigel 1:1. Tumor volume and body weights were monitored twice a week until the tumors reached ~ 150 mm³. Once the tumor burden was reached, mice were randomized into five groups—(1) vehicle (n = 15 mice), (2) Compound 1 (n = 15 mice), (3) venetoclax (n = 15 mice), (4) first combination of Compound 1 and Venetoclax (n = 15 mice), and (5) second combination of Compound 1 and venetoclax (n=15 mice)-and dosed as outlined in Table 9 below. For groups 4 and 5 wherein Compound 1 and venetoclax were co-administered on the same day, Compound 1 was dosed at T=0 hr and T=12 hr and venetoclax dosed at T=6 hr. At the end of study, tumor, plasma, and liver tissues were collected at the indicated timepoints post-last dose for PK and biomarker analyses. Compound 1 was formulated with 0.5% Methyl Cellulose/deionized water. Venetoclax was formulated with 60% Phosal 50, 30% PEG 400, and 10% ethanol and stored at 4° C.

TABLE 9

| Group | Cpd 1 Dose | Cpd 1 Schedule | Venetoclax Dose | Venetoclax Schedule | End of Study: Time Points for PK |
|---|---|---|---|---|---|
| 1 | VEHICLE - 60% Phosal 50 + 30% PEG 400+ 10% Ethanol | | | | End of Study- Pre, 1, 4, 7 & 24 hr (N=3/TP) |
| 2 | 30 mg/kg | BID | N/A | N/A | |
| 3 | N/A | N/A | 1 mg/kg | QD | |
| 4 | 30 mg/kg | BID | 1 mg/kg | QD | |
| 5 | 100 mg/kg | 2 days on, 1 off (BID) | 1 mg/kg | QD | |

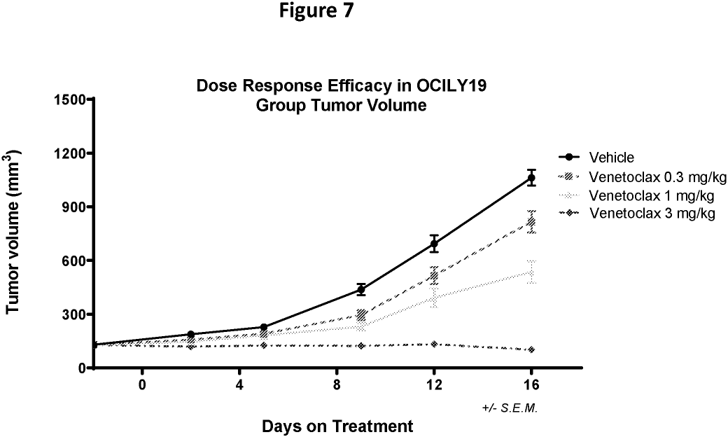

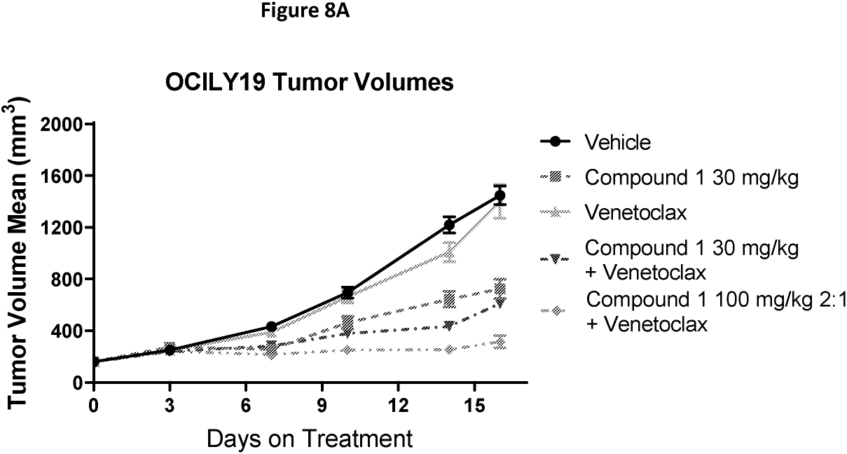

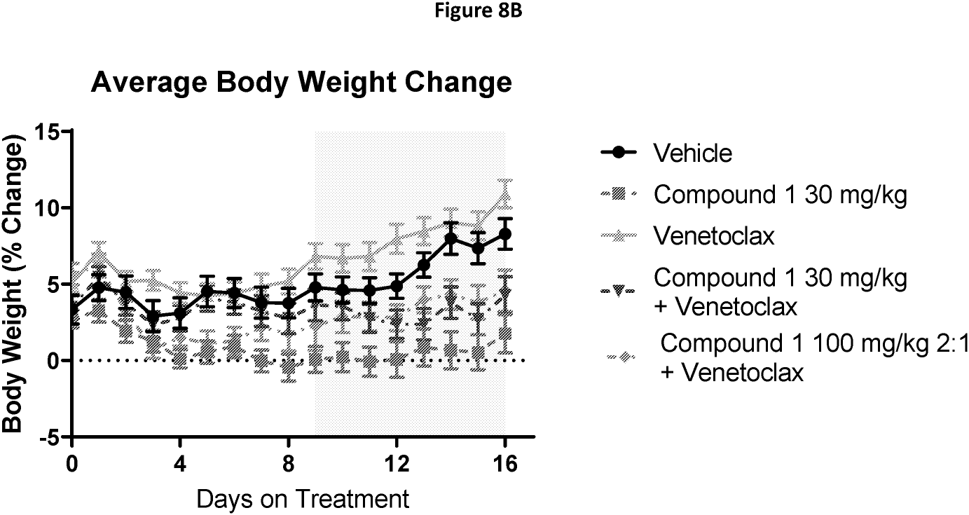

Animals treated with venetoclax alone at the 1 m/kg dose showed no effect. (FIG. 7; Table 10.) There was no difference in tumor growth inhibition between Compound 1 administered alone at 30 mg/kg BID and Compound 1 administered 30 mg/kg BID in combination with venetoclax. (FIG. 8A; Table 10.) The combination of Compound 1, given at 100 mg/kg on a holiday schedule (2 days on, 1 day off), with venetoclax showed a greater than 30% increase in tumor growth inhibition when compared to Compound 1 alone. (FIG. 8A; Table 10.) Each combination was well-tolerated. (FIG. 8B.)

TABLE 10

| Treatment | Tumor Growth Inhibition |
|---|---|
| Compound 1 (30 mg/kg BID) | 56% |
| Venetoclax (1 mg/kg) | 3.6% |
| Compound 1 (30 mg/kg BID) + Venetoclax (1 mg/kg) | 65% |
| Compound 1 (100 mg/kg holiday schedule) + Venetoclax (1 mg/kg) | 88% |

### Example 4

### Tolerability Study of Compound 1 With Azacitidine and Venetoclax

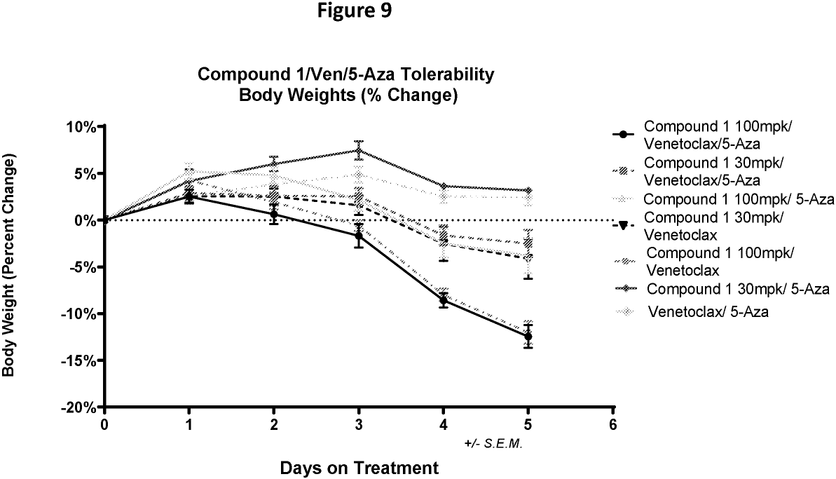

Based on the anti-tumor effects seen with Compound 1 in combination with venetoclax and with azacitidine, it was anticipated that adding azacitidine to the combination of Compound 1 and venetoclax would increase the tumor growth inhibition if the triple combination is tolerated. As shown in FIG. 9 (percent body weight change), combinations of Compound 1 and azacitidine were tolerated at all dose levels. Combinations of Compound 1, administered at 30 mg/kg, with venetoclax administered QD at 30 mg/kg or azacitidine administered Q3d at 5 mg/kg were tolerated, as well as the combination of all three agents. Combinations with Compound 1 given at 100 mg/kg on a BID schedule were not tolerated. These data demonstrate that Compound 1, venetoclax, and azacitidine can be combined at dose levels that have been shown to be effective in demonstrating anti-tumor growth activity.

The maximum tolerated dose of Compound 1 in combination with 5-azacitidine and venetoclax using varying dosing holidays or concentrations of Compound 1 can be further evaluated in female CB17 SCID mice by administering a total of 10 days of venetoclax (QD) at a single concentration in combination with 10 days of azacitidine (Q3D) and Compound 1 given continuously or on a dosing schedule of 3 days on and 4 days off for a total time period of 10 days (ending at the end of the “on” period). (Table 11; Dosing Schema.) Both Compound 1 and venetoclax are given orally and azacitidine given I.P., all at a concentration of 10 ml/kg in naive CB17 SCID mice. Body weights and clinical signs are observed daily.

TABLE 11

| Grp No. | Cpd 1 Dose | Cpd 1 Schedule | Venetoclax Dose | Venetoclax Schedule | 5-Azacitidine Dose | 5-Azacitidine Schedule |
|---|---|---|---|---|---|---|
| 1 | 100 mg/kg | 3 days on, 4 days off (BID) | 30 mg/kg | QD | 5 mg/kg | Q3D |
| 2 | 30 mg/kg | BID | 30 mg/kg | QD | 5 mg/kg | Q3D |
| 3 | 100 mg/kg | 3 on 4 off (BID) | N/A | NA | 5 mg/kg | Q3D |
| 4 | 30 mg/kg | BID | N/A | NA | 5 mg/kg | Q3D |
| 5 | 100 mg/kg | 3 on 4 off (BID) | 30 mg/kg | QD | NA | NA |
| 6 | 30 mg/kg | BID | 30 mg/kg | QD | NA | NA |
| 7 | NA | NA | 30 mg/kg | QD | 5 mg/kg | Q3D |
| 8 | NA | NA | 30 mg/kg | QD | 5 mg/kg | Q3D |

Dosing Schema

| T_{X} | Group | Day 1 | Day 2 | Day 3 | Day 4 | Day 5 | Day 6 | Day 7 | Day 8 | Day 9 | Day 10 |
|---|---|---|---|---|---|---|---|---|---|---|---|
| Compound 1100 mpk (3:4) | 1, 3, 5 | XX | XX | XX | | | | | XX | XX | XX |
| Compound 130 MPK BID | 2, 4, 6 | XX | XX | XX | XX | XX | XX | XX | XX | XX | XX |
| Veneteclax 30 mpk QD | 1, 2, 5, 6, 7, 8 | X | X | X | X | X | X | X | X | X | X |
| 5-Azacitidine Q3D | 1, 2, 3, 4, 7, 8 | | | X | | | | X | | | X |

What is claimed is:

1. A method for treating a cancer in a subject, comprising administering to the subject an effective amount of a first anti-cancer agent and an effective amount of at least one second anti-cancer agent, wherein the first anti-cancer agent is represented by Formula (I): 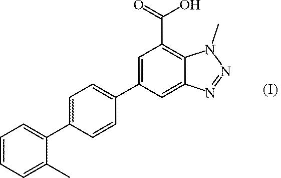 [SMILES: Cc1ccccc1-c1ccc(-c2cc(C(=O)O)c3c(c2)nnn3C)cc1]
or a pharmaceutically acceptable salt thereof; and wherein the at least one second anti-cancer agent is selected from the group consisting of a BH3 mimetic and a DNA demethylating agent, or a combination thereof.

2. The method of claim 1, wherein the cancer is a hematological malignancy.

3. The method of claim 1, wherein the cancer is selected from the group consisting of a leukemia and a lymphoma.

4. The method of claim 1, wherein the cancer is selected from the group consisting of acute myeloid leukemia (AML), multiple myeloma, B-prolymphocytic leukemia, acute lymphoblastic leukemia, chronic lymphocytic leukemia, Hodgkin’s disease, non-Hodgkin’s lymphoma, follicular lymphoma, anaplastic large cell lymphoma, mantle cell lymphoma, T-cell lymphoma, acute monocytic leukemia, B-cell lymphoma, diffuse mixed cell lymphoma, myelodysplastic syndrome, primary effusion lymphoma, erythroleukemia, chronic myeloid leukemia (CML), chronic myelomonocytic leukemia (CMML), double hit diffuse large B cell lymphoma, triple hit diffuse large B cell lymphoma, blastic plasmacytoid dendritic cell neoplasm, and small lymphocytic lymphoma (SLL).

5. The method of claim 1, wherein the cancer is selected from the group consisting of diffuse large B cell lymphoma, chemotherapy-resistant acute myeloid leukemia, and cytarabine-resistant acute myeloid leukemia.

6. The method of claim 1, wherein the cancer is selected from acute myeloid leukemia, multiple myeloma, B-prolymphocytic leukemia, acute lymphoblastic leukemia, chronic lymphocytic leukemia, Hodgkin’s disease, non-Hodgkin’s lymphoma, follicular lymphoma, diffuse large B cell lymphoma, anaplastic large cell lymphoma, mantle cell lymphoma, and T-cell lymphoma.

7. The method of claim 1, wherein the cancer is selected from acute myeloid leukemia, multiple myeloma, B-prolymphocytic leukemia, non-Hodgkin’s lymphoma, diffuse large B cell lymphoma, anaplastic large cell lymphoma, and mantle cell lymphoma.

8. The method of claim 1, wherein the cancer is selected from acute myeloid leukemia and diffuse large B-cell lymphoma.

9. The method of claim 1, wherein the at least one second anti-cancer agent is a combination of a BH3 mimetic and a DNA demethylating agent.

10. The method of claim 1, wherein the second anti-cancer agent is a BH3 mimetic selected from the group consisting of venetoclax, BGB-11417, navitoclax (ABT-263), LP-108, S55746, S65487, AT-101, APG-1252, obatoclax, APG2575, and BCL-201.

11-12. (canceled)

13. The method of any claim 1, wherein the second anti-cancer agent is a DNA demethylating agent selected from the group consisting of 5-azacitidine (azacitidine), 5-aza-2′-deoxycytidine (decitabine), 1-β-D-arabinofuranosylcytosine (cytarabine or ara-C), pseudoiso-cytidine (psi ICR), 5-fluoro-2′-deoxycytidine (FCdR), 2′-deoxy-2′,2′-difluorocytidine (gemcitabine), 5-aza-2′-deoxy-2′,2′-difluorocytidine; 5-aza-2′-deoxy-2′-fluorocytidine; 1-β-D-ribofuranosyl-2(1H)-pyrimidinone (Zebularine), 2′,3′-dideoxy-5-fluoro-3′-thiacytidine (emtriva), 2′-cyclocytidine (Ancitabine), 1-β-D-arabinofuranosyl-5-azacytosine (fazarabine or ara-AC), 6-azacitidine (6-aza-CR), 5,6-dihydro-5-azacitidine (dH-aza-CR), N4 pentyloxy-carbonyl-5′-deoxy-5-fluorocytidine (capecitabine), N4 octadecyl-cytarabine, and elaidic acid cytarabine.

14-17. (canceled)

18. The method of claim 1, wherein the first anti-cancer agent and the at least one second anti-cancer agent are administered simultaneously in the same or different formulations.

19. The method of claim 1, wherein the first anti-cancer agent and the at least one second anti-cancer agent are administered sequentially.

20. The method of claim 1, wherein the first anti-cancer agent is the tris(hydroxymethyl)aminomethane salt of the compound of Formula (I).

21. The method of claim 1, wherein the first anti-cancer agent is the sodium salt of the compound of Formula (I).

22. A pharmaceutical composition comprising a first anti-cancer agent and at least one second anti-cancer agent, wherein the pharmaceutical composition additionally comprises an excipient, wherein the first anti-cancer agent is represented by Formula (I): 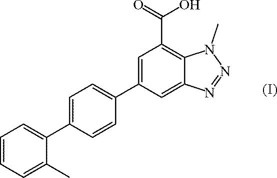 [SMILES: Cc1ccccc1-c1ccc(-c2cc(C(=O)O)c3c(c2)nnn3C)cc1]
or a pharmaceutically acceptable salt thereof; and wherein the at least one second anti-cancer agent is selected from the group consisting of a BH3 mimetic and a DNA demethylating agent, or a combination thereof.

23. The pharmaceutical composition of claim 22, wherein the at least one second anti-cancer agent is a combination of a BH3 mimetic and a DNA demethylating agent.

24. The pharmaceutical composition of claim 22, wherein the at least one second anti-cancer agent is a BH3 mimetic selected from the group consisting of venetoclax, BGB-11417, navitoclax (ABT-263), LP-108, S55746, S65487, AT-101, APG-1252, obatoclax, APG2575, and BCL-20.

25-26. (canceled)

27. The pharmaceutical composition of claim 22, wherein the second anti-cancer agent is a DNA demethylating agent selected from the group consisting of 5-azacitidine (azacitidine), 5-aza-2′-deoxycytidine (decitabine), 1-β-D-arabinofuranosylcytosine (cytarabine or ara-C), pseudoiso-cytidine (psi ICR), 5-fluoro-2′-deoxycytidine (FCdR), 2′-deoxy-2′,2′-difluorocytidine (gemcitabine), 5-aza-2′-deoxy-2′,2′-difluorocytidine; 5-aza-2′-deoxy-2′-fluorocytidine; 1-β-D-ribofuranosyl-2(1H)-pyrimidinone (Zebularine), 2′,3′-dideoxy-5-fluoro-3′-thiacytidine (emtriva), 2′-cyclocytidine (Ancitabine), 1-β-D-arabinofuranosyl-5-azacytosine (fazarabine or ara-AC), 6-azacitidine (6-aza-CR), 5,6-dihydro-5-azacitidine (dH-aza-CR), N4 pentyloxy-carbonyl-5′-deoxy-5-fluorocytidine (capecitabine), N4 octadecyl-cytarabine, and elaidic acid cytarabine.

28-31. (canceled)

32. The pharmaceutical composition of claim 22, wherein the first anti-cancer agent is the tris(hydroxymethyl)aminomethane salt or the sodium salt of the compound of Formula (I).

33. (canceled)
